(19) United States
(12) Patent Application Publication
(10) Pub. No.: US 2022/0211691 A1
(43) Pub. Date: Jul. 7, 2022

(54) TREATMENT OF DRY EYE DISEASE

(71) Applicant: Aldeyra Therapeutics, Inc., Lexington, MA (US)

(72) Inventors: Todd Brady, Carlisle, MA (US); Stephen Gitu Machatha, Wilmington, MA (US)

(21) Appl. No.: 17/654,969

(22) Filed: Mar. 15, 2022

Related U.S. Application Data
(63) Continuation of application No. PCT/US2022/011604, filed on Jan. 7, 2022.
(60) Provisional application No. 63/265,773, filed on Dec. 20, 2021.
(60) Provisional application No. 63/134,611, filed on Jan. 7, 2021.

(51) Int. Cl. A61K 31/47; A61K 9/16

(52) U.S. Cl. CPC A61K 31/47; A61K 9/1652

(57) ABSTRACT

The present invention provides a reproxalap ophthalmic solution, and methods of using the same for treating dry eye disease.

### CROSS-REFERENCE TO RELATED APPLICATIONS

This application is a continuation of International Application No. PCT/US2022/011604, filed Jan. 7, 2022, which claims the benefit under 35 U.S.C. § 119(e) of U.S. Provisional Patent Application Ser. No. 63/134,611, filed on Jan. 7, 2021, and U.S. Provisional Patent Application Ser. No. 63/265,773, filed on Dec. 20, 2021; the entirety of each of which is hereby incorporated by reference in its entirety.

### TECHNICAL FIELD

The present invention relates to methods of treating dry eye disease (DED) using ophthalmic solutions comprising reproxalap, or a pharmaceutically acceptable salt thereof.

### BACKGROUND

Dry eye disease is a complex disease that results in ocular discomfort, visual disturbance, and tear film instability, which create the potential for damage to the ocular surface. It is characterized by increased osmolarity of the tear film and inflammation of the ocular surface. Estimates of the prevalence of dry eye disease vary considerably, depending on the criteria used to define the disease, but in the United States (U.S.), it has been estimated that as many as 20 million adults in the U.S. have dry eye disease. It has been projected that the number of patients afflicted with dry eye disease will continue to increase (Schaumberg, Advances in Experimental Medicine and Biology, 2002, 506:989-98; Schaumberg, American Journal of Ophthalmology, 2003, 136:318-26; Schaumberg, Archives of Ophthalmology, 2009, 127:763-8). With the aging population in the U.S. and other countries of the developed world, and increasing computer use, dry eye disease is expected to become more prevalent. Thus, finding a treatment is becoming more important (Brewitt, Survey of Ophthalmology, 2001, 45 Suppl 2:S199-202).

Aldehydes are reactive organic molecules that bind to proteins, carbohydrates, lipids and nucleic acids (Esterbauer, Free Radical Biology and Medicine, 1991, 11(1):81-128). Free aldehydes—aldehydes not sequestered or otherwise protected in specific metabolic processes—can be toxic, and aldehyde binding to cellular constituents can lead to inflammation (Yadav, Oxidative Medicine and Cellular Longevity, 2013, Volume 2013, Article ID 690545), molecular dysfunction (O'Brien, Critical Reviews in Toxicology, 2005, 35(7):609-62), and the accumulation of indigestible metabolites, such as lipofuscin components in the retina (Boyer, J Biol Chem., 2012, 287:22276-86).

In biological systems, aldehydes are formed by a variety of processes, including the oxidation of alcohols, polyamine and glucose metabolism, and oxidative stress. In some disease states, aldehyde concentrations may be increased. Increases in aldehyde concentrations, particularly malonyldialdehyde (MDA), which is thought to be most commonly derived from lipid peroxidation, has been described in a variety of inflammatory ocular diseases, including pterygium, Behcet's Disease, Sjögren's Syndrome, anterior uveitis, and dry eye disease (Sandikci, Acta Dermato-Venereologica, 2003, 83(5): 342-6; Cejkova, Histology and Histopathology, 2007, 22(9):997-1003; Balci, Molecular Vision, 2011, 17: 443-7; Turk, Ocular Immunology and Inflammation, 2014, 22(2):127-32; Choi, Current Eye Research, 2016, 41(9): 1143-9).

### SUMMARY

In one aspect, the present disclosure provides a method of treating dry eye disease by administering a therapeutically effective amount of an ophthalmic solution comprising reproxalap. The present disclosure shows that administration of reproxalap results in immediate and durable improvements in treating dry eye disease.

Accordingly, in some embodiments, the present disclosure provides a method of treating dry eye disease in a subject, comprising topically administering a therapeutically effective amount of an ophthalmic solution comprising reproxalap, or a pharmaceutically acceptable salt thereof, to an eye of a subject in need thereof prior to an expected or certain exposure to one or more conditions that initiate dry eye disease or its symptoms, and topically administering a therapeutically effective amount of the ophthalmic solution to the eye subsequent to exposure to the conditions that initiate dry eye disease or its symptoms.

In some embodiments, the ophthalmic solution is administered one day or less and/or immediately prior to an expected or certain exposure to one or more conditions that initiate dry eye disease or its symptoms. In some embodiments, the initial dose administered immediately prior to an expected or certain exposure to one or more conditions that initiate dry eye disease or its symptoms is followed by administration of the ophthalmic solution about 45 min or 60 min after the initial dose or after the exposure to one or more conditions that initiate dry eye disease or its symptoms.

In another embodiment, the present disclosure provides a method of treating dry eye disease by administering a therapeutically effective amount of an ophthalmic solution comprising reproxalap at or immediately after the onset of the symptoms of dry eye disease. In some embodiments, the initial dose administered at or immediately after the onset of the symptoms of dry eye disease is followed by administration of the ophthalmic solution to the eye about 45 min or 60 min after the initial dose or onset of symptoms of dry eye disease.

In some embodiments, in treatments prior to or after the onset of symptoms of dry eye disease, an ophthalmic solution disclosed herein is administered four times a day (QID). In some embodiments, an ophthalmic solution disclosed herein is administered three times a day (TID). In some embodiments, an ophthalmic solution disclosed herein is administered two times a day (BID) or once a day. In some embodiments, an ophthalmic solution disclosed herein is administered as needed (PRN).

In some embodiments, an ophthalmic solution disclosed herein is administered four times a day (QID) in an initiation phase followed by administration of fewer than four times a day, for example, one, two, or three times a day. In some embodiments, an ophthalmic solution disclosed herein is administered four times a day (QID) in an initiation phase followed by administration two times a day (BID).

In some embodiments, the QID administration in the initiation phase produces an improvement in tear quantity and/or quality in the eye of the subject. In some embodiments, the improvement is measured using Schirmer's Test. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below. In some embodiments, the patient originally exhibited a Schirmer's Test score of ≤10 mm and ≥1 mm before treatment in the method provided by the present invention. In some embodiments, the Schirmer's Test score improves to at least 10 mm, 11 mm, 12 mm, 13 mm, 14 mm, 15 mm, or more than 15 mm after treatment with the ophthalmic solution. In some embodiments, the Schirmer's Test result improves by at least about 2 mm, 3 mm, 4 mm, 5 mm, 6 mm, 7 mm, 8 mm, or greater than 8 mm after treatment. In some embodiments, the Schirmer's Test result improves by an increase of about 8 mm, about 9 mm, about 10 mm, about 11 mm, about 12 mm, about 13 mm, about 14 mm, about 15 mm, or about 16 mm after treatment. In some embodiments, the Schirmer's Test score improves to at least about 20 mm (i.e., a normal test result). In some embodiments, the Schirmer's Test score improves to about 20 mm, about 21 mm, about 22 mm, about 23 mm, about 24 mm, about 25 mm, or about 26 mm or more.

In some embodiments, the QID administration in the initiation phase produces an improvement in ocular redness. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

In some embodiments, the QID administration in the initiation phase produces an improvement in ocular dryness. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

In some embodiments, the QID administration in the initiation phase produces an improvement in ocular itch. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

In some embodiments, the present invention provides a method of treating dry eye disease, comprising administering a therapeutically effective amount of an ophthalmic solution comprising reproxalap, wherein the ophthalmic solution is administered to an eye of the subject in need thereof at or immediately after the onset of symptoms of dry eye disease.

In some embodiments, the method further comprises administering the ophthalmic solution to the eye of the subject 45 min or 60 min following the onset of symptoms of symptoms of dry eye disease.

In some embodiments, the ophthalmic solution is administered four times a day (QID) in an initiation phase one day prior to an expected or certain exposure to one or more conditions that initiate dry eye disease or its symptoms followed by administration two times a day (BID).

In some embodiments, the ophthalmic solution is administered four times a day (QID) in an initiation phase one day prior to an expected or certain exposure to one or more conditions that initiate dry eye disease or its symptoms followed by administration as needed (PRN).

In some embodiments, the method further comprises assessing effectiveness of treatment by determining one or more of ocular dryness, redness, itchiness, tearing, burning, stinging, grittiness, cloudy vision, sensitivity to environment, and level of RASP in tears.

In some embodiments, assessing effectiveness of treatment is determined by measuring level of RASP in tears.

In some embodiments, the effectiveness is reducing level of RASP to levels present in normal eyes.

In some embodiments, the method produces an improvement in tear quantity and/or quality in the eye of the subject.

In some embodiments, the improvement is measured using Schirmer's Test.

In some embodiments, the method produces an improvement in ocular redness.

In some embodiments, the method produces an improvement in ocular dryness.

In some embodiments, the method produces an improvement in ocular itchiness.

In some embodiments, the improvement is measured as a statistically significant (p value of 0.05 or less) change in ocular dryness, redness, itchiness, tearing, burning, stinging, grittiness, cloudy vision, sensitivity to environment, and/or level of RASP in tears in a group of subjects who were administered the ophthalmic solution under similar conditions.

### BRIEF DESCRIPTION OF THE DRAWINGS

FIG. 1 shows Day 2 results of Ocular Dryness Score (VAS) in subjects exposed to dry eye chamber.

FIG. 2 shows Day 2 results of Mean Ocular Discomfort Score in subjects exposed to dry eye chamber.

FIG. 3 shows Day 2 results of Mean Ocular Redness Score in subject exposed to dry eye chamber.

FIG. 4 shows Day 1 results of Schirmer's test for patients who were administered topical ophthalmic reproxalap four times daily in the Phase 3 TRANQUILITY trial in dry eye disease. MMRM=mixed effect model of repeated measures. Estimates reflect change from baseline. Schirmer test performed on Day 1 after the first, second, and fourth doses of test article for Phase 2, Phase 3 TRANQUILITY run-in, and Phase 3 TRANQUILITY clinical trials, respectively.

FIG. 5 shows Schirmer's test change from baseline data for post-hoc responders who showed a ≥10 mm improvement from baseline. ^{‡}Generalized estimating equation analysis controlling for baseline, pre/post dose, and site. MMRM=mixed effect model of repeated measures. SEM=standard error of the mean. Schirmer test administered before and after fourth dose on Day 1. Graph horizontal axis values offset for clarity.

FIG. 6 shows Ocular Dryness Symptom Score and Ocular Itching Symptom Score data for patients who were administered topical ophthalmic reproxalap four times daily in the Phase 3 TRANQUILITY trial in dry eye disease.

FIG. 7 shows mean adduct concentrations in tears from D/C subjects collected at Visit 1 and normal human tears (NHT). Normal human tears diluted 20-fold had an average calculated MDA adduct concentration of 2,266 pmol/mL. Tears from D/C DES subjects had a mean MDA adduct concentration of 7,798 pmol/mL, a 3.4 fold increase in relative to NHT.

FIG. 8 shows mean MDA adduct concentrations in tears from 37 DES subject who completed the trial, at Visit 1 (baseline, before treatment) and Visit 3 (after 28 days of treatment with reproxalap ophthalmic solution. Tears collected at Visit 1 had a mean MDA adduct concentration of 14,943 pmol/mL, which is significantly higher than the mean MDA adduct concentration of 11,566 pmol/mL in tears collected from all subjects at Visit 3.

FIG. 9 shows mean MDA adduct concentrations in tears from 37 DES subject who completed the trial, at Visit 1 (baseline, before treatment) and Visit 3 (after 28 days of treatment with 0.1% w/v reproxalap ophthalmic solution. Tears from study subjects treated with reproxalap ophthalmic solution (0.1%) had a mean MDA adduct concentration of 14,287 pmol/mL at Visit 1, compared to 11,028 pmol/mL at Visit 3, which corresponds to a 23% reduction in MDA adduct levels after treatment.

FIG. 10 shows mean MDA adduct concentrations in tears from 37 DES subject who completed the trial, at Visit 1 (baseline, before treatment) and Visit 3 (after 28 days of treatment with 0.5% w/v reproxalap ophthalmic solution). DES study subjects showed a 26% reduction in MDA adduct concentration at Visit 3 compared to Visit 1.

FIG. 11 shows reduction in HNE-protein adduct levels in DED patient tears (run-in Phase 2/3 data, see Example 3). The data show Day 1 and Day 2 pre/post dose results (pre-dose to post-dose change) in HNE-protein adduct levels (pg/mL) in the patients dosed with either vehicle or reproxalap. Day 1 dose is first dose of Day 1. Day 2 dose is dose post chamber. Tear collections taken approximately 10 minutes before and after dosing.

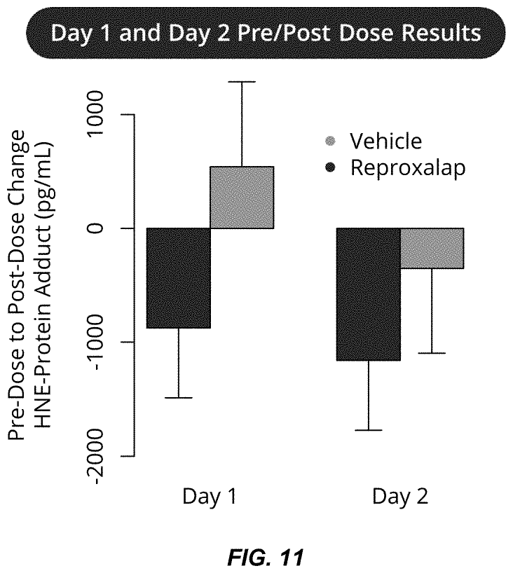

FIG. 12 shows the mean of reduction in HNE-protein adduct levels in DED patient tears (run-in Phase 2/3 data, see Example 3). P values by group represent difference from 0 (no change). Means represent average of the two doses where tear RASP were assessed before and after dosing. HNE=4-hydroxynonenal ELISA of protein adducts. Tear RASP levels from the Phase 3 clinical trial run-in cohort were reduced after single doses of the novel RASP inhibitor reproxalap, as assessed by enzyme-linked immunosorbent assay (ELISA) of 4-hydroxynonenal protein adducts (HNE), a RASP selected based on results from a natural history study of dry eye patients. For subjects with sufficient tear volumes for analysis, across the two doses where tear RASP levels were assessed before and after drug administration, HNE levels declined by an average of 1018 picograms/milliliter (pg/mL) in reproxalap-treated patients (n=9) versus an increase of 32 pg/mL in vehicle-treated patients (n=7).

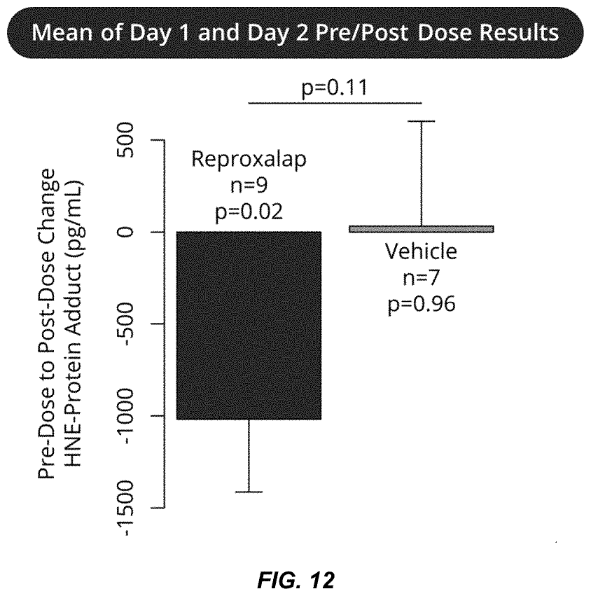

FIG. 13 shows MDA concentration in tears of pooled reproxalap groups. At baseline and after completion of treatment, MDA was measured in the tears of dry eye disease patients by ELISA in tears extracted through capillary. Both eyes were pooled per patient. A standard curve was generated, and a 1:60 dilution was established as optimal using 3 mL of tears per patient. Above- and below median percentage MDA reduction subgroups were compared using 2-way t tests and 1-way t tests versus 0 (no change from baseline). (A) Within-participant tear MDA adduct levels before treatment were compared with tear MDA adduct levels after treatment. (B) Total lissamine green staining scores at day 28 in participants with below median MDA adduct reduction after treatment were compared with those of participants with above-median MDA adduct reduction after treatment. MDA=malondialdehyde.

FIG. 14 shows (top) the initial results of a HABA/avidin/biotin assay for measuring levels of HNE-protein adducts in biological samples; and (bottom) the initial results of a streptavidin plate/lysozyme antibody assay for measuring levels of HNE-protein adducts in biological samples.

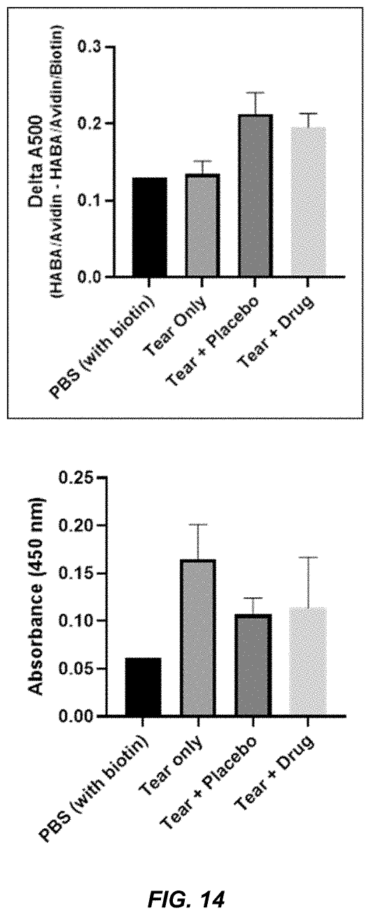

### DETAILED DESCRIPTION

### 1. Detailed Description of Embodiments

Reproxalap has the following structural formula:

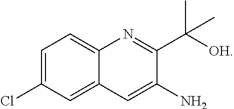 [SMILES: CC(C)(O)c1nc2ccc(Cl)cc2cc1N]

Without wishing to be bound by any particular theory, reproxalap functions as an aldehyde sequestering agent, or “trap,” which binds rapidly to aldehydes and forms a stable cyclic product, thereby sequestering the aldehyde. An ophthalmic solution of reproxalap formulated with cyclodextrin shows efficacy in treating dry eye disease. In the present disclosure, reproxalap is shown to provide immediate and durable effectiveness in treating dry eye disease or its symptoms. Moreover, treatment prior to an expected or certain exposure to one or more conditions that initiate dry eye disease or its symptoms is shown to provide durable improvement in dry eye disease symptoms.

Accordingly, in some embodiments, the present disclosure provides a method of treating dry eye disease by administering a therapeutically effective amount of an ophthalmic solution comprising reproxalap prior to an expected or certain exposure to one or more conditions that initiate dry eye disease or its symptoms. In some embodiments, the ophthalmic solution is administered immediately prior to an expected or certain exposure to one or more conditions that initiate dry eye disease or its symptoms. In some embodiments, the initial dose administered immediately prior to an expected or certain exposure to one or more conditions that initiate dry eye disease or its symptoms is followed by administration of the ophthalmic solution about 45 min or 60 min after the initial dose or after the exposure to one or more conditions that initiate dry eye disease or its symptoms.

### 2. Definitions

The general terms used herein are defined with the following meanings, unless explicitly stated otherwise.

The term “comprising” and “including” are used herein in their open-ended and non-limiting sense unless otherwise noted. It is to be further understood that where descriptions of various embodiments use the term “comprising” or “including,” those skilled in the art would understand that in some specific instances, an embodiment can be alternatively described using language “consisting essentially of” or “consisting of.”

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “pharmaceutically acceptable” is defined herein to refer to those compounds, biologic agents, materials, compositions and/or dosage forms, which are, within the scope of sound medical judgment, suitable for contact with the tissues a subject e.g., a mammal or human, without excessive toxicity, irritation allergic response and other problem complications commensurate with a reasonable benefit/risk ratio.

The term “treating” or “treatment” as used herein comprises a treatment relieving, reducing or alleviating at least one symptom in a subject or affecting a delay of progression of a disease, condition and/or disorder. For example, treatment can be the diminishment of one or several signs or symptoms of a disorder or complete eradication of a disorder. Within the meaning of the present invention, the term “treat” also denotes to arrest, delay the onset (e.g., the period prior to clinical manifestation of a disease) and/or reduce the risk of developing or worsening a disease.

The term “subject” or “patient” as used herein includes animals, such as mammals, e.g., humans, dogs, cows, horses, pigs, sheep, goats, cats, mice, rabbits, rats and transgenic non-human animals. In some embodiments, the subject is a human.

The term “about” or “approximately” shall have the meaning of within 10% of a given value or range. In some embodiments, the term “about” refers to within 10%, 9%, 8%, 7%, 6%, 5%, 4%, 3%, 2%, or 1% of a given value.

The term “w/v” as used herein refers to “gram/mL” (weight over volume), which is a concentration unit. For example, 7% w/v is equivalent to 70 mg/mL.

As provided by FDA guidance, Sponsors developing drugs for the treatment of dry eye should consider the following regarding efficacy:

In general, safety and efficacy should be demonstrated in at least two adequate and well-controlled, multicenter independent trials.

FDA recommends that the sponsor demonstrate one of the following:
A statistically significant difference between the investigational treatment and vehicle for at least one objective prespecified sign of dry eye (mean group score of test versus vehicle) and at least one subjective prespecified symptom of dry eye (mean group score), or
A statistically significant difference between the percentage of patients achieving a complete resolution of corneal staining, or
A statistically significant difference between the percentage of patients achieving a 10-millimeter increase or more in Schirmer's tear test scores.

If a sign and a symptom are used to demonstrate efficacy, FDA recommends a number of different endpoints for an objective sign or subjective symptom (see bullet points below).

Signs of dry eye include, but are not limited to, corneal staining, conjunctival staining, decreased tear breakup time, and decreased Schirmer's tear test score (with or without anesthesia).

Symptoms of dry eye include, but are not limited to, blurred vision, light sensitivity, sandy or gritty feeling, ocular irritation, ocular pain or discomfort, and ocular itching. Subjects can self-identify their own term for ocular discomfort, which can be used in place of any other term.

A subjective symptom improvement can also be demonstrated by showing a statistically significant difference between the percentage of patients achieving a complete resolution of the symptom. FDA does not recommend the use of anything less than complete resolution (complete clearing of a sign or symptom) for a responder analysis.

Efficacy for a sign and efficacy for a symptom do not have to be demonstrated in the same clinical trial, but each should be demonstrated in more than one clinical trial.

### 3. Ophthalmic Solutions

An ophthalmic solution of the invention comprises reproxalap, or a pharmaceutically acceptable salt thereof, at a concentration suitable for effectively treating dry eye disease, in particular without causing severe or intolerable adverse effects. In some embodiments, the present invention provides an ophthalmic solution comprising about 0.1% to 0.5% w/v reproxalap, or a pharmaceutically acceptable salt thereof, and a pharmaceutically acceptable excipient. In some embodiments, the excipient comprises a cyclodextrin, such as sulfobutylether β-cyclodextrin (SBECD) or hydroxypropyl β-cyclodextrin.

In some embodiments, an ophthalmic solution comprises reproxalap and a cyclodextrin excipient in a ratio of less than 1:2.1 on a mole:mole basis. In some embodiments, the ratio of reproxalap and cyclodextrin is about 1:2.1 to about 1:25 ratio on a mole:mole basis. In some embodiments, the ratio is about 1:2.2 to 1:20, 1:2.5 to 1:20, 1:2.5 to 1:10, 1:2.75 to 1:10, 1:3 to 1:8, 1:3.5 to 1:7, 1:4 to 1:6, or 1:4 to 1:5 in a mole:mole basis. In some embodiments, the ratio is about 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:6, 1:7, 1:8, 1:9, 1:10, 1:12, 1:15, 1:20, or 1:25 on a mole:mole basis.

In some embodiments, the cyclodextrin excipient is one of those described herein, such as sulfobutylether β-cyclodextrin (SBECD). The average degree of substitution of the SBECD is about 6.5.

In some embodiments, the ratio of reproxalap to the excipient is about 1:2.1 or less on a mole:mole basis.

In some embodiments, the excipient is a cyclodextrin and the ratio of reproxalap to the excipient is about 1:2.1 to about 1:25 on a mole:mole basis.

In some embodiments, the excipient is a cyclodextrin and the ratio of reproxalap to the excipient is about 1:2 to about 1:5 on a mole:mole basis.

In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is about 0.5% w/v or less and about 0.1% w/v or greater. In some embodiments, the ophthalmic solution comprises about 0.15 to about 0.45% w/v reproxalap, or a pharmaceutically acceptable salt thereof, and a pharmaceutically acceptable excipient. In some embodiments, the ophthalmic solution comprises about 0.2 to about 0.4% w/v reproxalap, or a pharmaceutically acceptable salt thereof, and a pharmaceutically acceptable excipient. In some embodiments, the ophthalmic solution comprises about 0.21 to about 0.35% w/v reproxalap, or a pharmaceutically acceptable salt thereof, and a pharmaceutically acceptable excipient. In some embodiments, the ophthalmic solution comprises about 0.22 to about 0.3% w/v reproxalap, or a pharmaceutically acceptable salt thereof, and a pharmaceutically acceptable excipient. In some embodiments, the ophthalmic solution comprises about 0.22 to about 0.29% w/v reproxalap, or a pharmaceutically acceptable salt thereof, and a pharmaceutically acceptable excipient. In some embodiments, the ophthalmic solution comprises about 0.25% w/v reproxalap, or a pharmaceutically acceptable salt thereof, and a pharmaceutically acceptable excipient. In some embodiments, the ophthalmic solution comprises about 0.25% w/v reproxalap and a pharmaceutically acceptable excipient selected from a cyclodextrin. In some embodiments, the ophthalmic solution comprises about 0.5% w/v reproxalap and a pharmaceutically acceptable excipient selected from a cyclodextrin.

In some embodiments, the present invention provides an ophthalmic solution comprising less than 0.5% w/v reproxalap, or a pharmaceutically acceptable salt thereof, and a pharmaceutically acceptable excipient. In some embodiments, the present invention provides an ophthalmic solution comprising at least 0.1% w/v reproxalap, or a pharmaceutically acceptable salt thereof, and a pharmaceutically acceptable excipient. In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is less than 0.5% w/v and 0.1% w/v or greater.

In some embodiments, reproxalap, or a pharmaceutically acceptable salt thereof, in an ophthalmic solution of the invention is at a concentration of less than 0.45% w/v and at least 0.1% w/v. In some embodiments, reproxalap, or a pharmaceutically acceptable salt thereof, in an ophthalmic solution of the invention is at a concentration of less than 0.4% w/v and at least 0.1% w/v. In some embodiments, reproxalap, or a pharmaceutically acceptable salt thereof, in an ophthalmic solution of the invention is at a concentration of less than 0.35% w/v and at least 0.1% w/v. In some embodiments, reproxalap, or a pharmaceutically acceptable salt thereof, in an ophthalmic solution of the invention is at a concentration of less than 0.3% w/v and at least 0.1% w/v. In some embodiments, reproxalap, or a pharmaceutically acceptable salt thereof, in an ophthalmic solution of the invention is at a concentration of less than 0.25% w/v and more than 0.1% w/v. In some embodiments, reproxalap, or a pharmaceutically acceptable salt thereof, in an ophthalmic solution of the invention is at a concentration of less than 0.2% w/v and at least 0.1% w/v. In some embodiments, reproxalap, or a pharmaceutically acceptable salt thereof, in an ophthalmic solution of the invention is at a concentration of less than 0.15% w/v and at least 0.1% w/v.

In some embodiments, reproxalap, or a pharmaceutically acceptable salt thereof, in an ophthalmic solution of the invention is at a concentration of 0.5% w/v or less and at least 0.15% w/v. In some embodiments, reproxalap, or a pharmaceutically acceptable salt thereof, in an ophthalmic solution of the invention is at a concentration of 0.5% w/v or less and at least 0.2% w/v. In some embodiments, reproxalap, or a pharmaceutically acceptable salt thereof, in an ophthalmic solution of the invention is at a concentration of 0.5% w/v or less and at least 0.25% w/v. In some embodiments, reproxalap, or a pharmaceutically acceptable salt thereof, in an ophthalmic solution of the invention is at a concentration of 0.5% w/v or less and at least 0.3% w/v. In some embodiments, reproxalap, or a pharmaceutically acceptable salt thereof, in an ophthalmic solution of the invention is at a concentration of 0.5% w/v or less and at least 0.35% w/v. In some embodiments, reproxalap, or a pharmaceutically acceptable salt thereof, in an ophthalmic solution of the invention is at a concentration of 0.5% w/v or less and at least 0.4% w/v. In some embodiments, reproxalap, or a pharmaceutically acceptable salt thereof, in an ophthalmic solution of the invention is at a concentration of 0.5% w/v or less and at least 0.45% w/v.

In some embodiments, reproxalap, or a pharmaceutically acceptable salt thereof, in an ophthalmic solution of the invention is at a concentration of about 0.1% to 0.5%, 0.15% to 0.45% w/v, 0.15% to 0.4% w/v, 0.15% to 0.35% w/v, 0.15% to 0.3% w/v, 0.15% to 0.25% w/v, or 0.15% to 0.2% w/v. In some embodiments, reproxalap, or a pharmaceutically acceptable salt thereof, in an ophthalmic solution of the invention is at a concentration of 0.2% to 0.45% w/v, 0.2% to 0.4% w/v, 0.2% to 0.35% w/v, 0.2% to 0.3% w/v, or 0.2% to 0.25% w/v. In some embodiments, reproxalap, or a pharmaceutically acceptable salt thereof, in an ophthalmic solution of the invention is at a concentration of 0.25% to 0.45% w/v, 0.25% to 0.4% w/v, 0.25% to 0.35% w/v, or 0.25% to 0.3% w/v. In some embodiments, reproxalap, or a pharmaceutically acceptable salt thereof, in an ophthalmic solution of the invention is at a concentration of 0.3% to 0.45% w/v or 0.3% to 0.4% w/v.

In some embodiments, reproxalap, or a pharmaceutically acceptable salt thereof, in an ophthalmic solution of the invention is at a concentration of about 0.1% w/v, 0.15% w/v, about 0.2% w/v, about 0.25%, about 0.30% w/v, about 0.35% w/v, about 0.4% w/v, about 0.45% w/v, or about 0.5% w/v.

In some embodiments, as further described herein, the foregoing concentrations of reproxalap can be selected and applied to treatment regimen that includes an initiation phase, an exacerbation phase, and/or a maintenance phase.

In some embodiments, a pharmaceutically acceptable excipient in an ophthalmic solution of the invention is a cyclodextrin. In some embodiments, a cyclodextrin is α-, β- and γ-cyclodextrin. In some embodiments, a cyclodextrin is a pharmaceutically acceptable derivative of a cyclodextrin, including, but not limited to, the hydroxyalkyl derivatives of α-, β- and γ-cyclodextrin (especially the hydroxyethyl and hydroxypropyl derivatives of β-cyclodextrin and γ-cyclodextrin), randomly methylated β-cyclodextrin, sulfobutylether β-cyclodextrin, sulfobutylether γ-cyclodextrin, and the so-called branched β- and γ-cyclodextrin derivatives such as glucosyl-β-cyclodextrin and glucosyl-γ-cyclodextrin. The natural cyclodextrins are either used alone or in a mixture of two or more cyclodextrins, by way of non-limiting example, a mixture of the γ-cyclodextrin and the more water-soluble hydroxypropyl γ-cyclodextrin, or γ-cyclodextrin and sulfobutylether γ-cyclodextrin, or β-cyclodextrin and hydroxypropyl-β-cyclodextrin, or β-cyclodextrin and sulfobutylether β-cyclodextrin.

In some embodiments, a cyclodextrin in an ophthalmic solution of the invention is at a concentration of 0 to 20% w/v. In some embodiments, a cyclodextrin in an ophthalmic solution of the invention is at a concentration of 1 to 18% w/v, 1 to 16% w/v, 1 to 14% w/v, 2 to 12% w/v, 4 to 10% w/v, 5 to 9% w/v, or 6 to 8% w/v. In some embodiments, the cyclodextrin in an ophthalmic solution of the invention is at a concentration of 7% to 11% w/v. In some embodiments, a cyclodextrin in an ophthalmic solution of the invention is at a concentration of about 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v.

In some embodiments, a pharmaceutically acceptable excipient in an ophthalmic solution of the invention is sulfobutylether-β-cyclodextrin, in particular at any of the specified concentrations and ranges of concentrations above, such as about 7% w/v. In some embodiments, a pharmaceutically acceptable excipient in an ophthalmic solution of the invention is hydroxypropyl-β-cyclodextrin, in particular at any of the specified concentrations and ranges of concentrations specified above, such as about 7% w/v.

In some embodiments, the ophthalmic solution comprises about 0.2% to 0.4% w/v reproxalap and about 7% to 25% w/v of a cyclodextrin excipient such as SBECD. In some embodiments, the ophthalmic solution comprises about 0.2%, 0.25%, 0.3%, 0.35%, or 0.4% w/v reproxalap and about 7% to 25% w/v of a cyclodextrin excipient such as SBECD.

In some embodiments, the ophthalmic solution comprises about 0.25% w/v reproxalap and about 4.7% to about 25% w/v of a cyclodextrin excipient such as SBECD.

In some embodiments, the ophthalmic solution comprises about 0.25% w/v reproxalap and about 7% to 25% w/v of a cyclodextrin excipient such as SBECD.

In some embodiments, the ophthalmic solution comprises about 0.25% w/v reproxalap and about 4.75% to about 11% w/v of a cyclodextrin excipient such as SBECD.

In some embodiments, the ophthalmic solution comprises about 0.5% w/v reproxalap and about 9.5% to about 11% w/v of a cyclodextrin excipient such as SBECD. In some embodiments, the ratio of API to SBECD is about a mole of API per 2 moles of SBECD.

In some embodiments, the ophthalmic solution comprises about 0.25% w/v reproxalap and about 7% w/v of a cyclodextrin excipient such as SBECD. In some embodiments, the ratio of API to SBECD is about a mole of API per 3 moles SBECD.

In some embodiments, the ophthalmic solution comprises about 0.25% w/v reproxalap and about 11% w/v of a cyclodextrin excipient such as SBECD. In some embodiments, the ratio of API to SBECD is about a mole of API per 5 moles SBECD.

In some embodiments, an ophthalmic solution of the invention comprises a pharmaceutically acceptable buffering agent. In some embodiments, a pharmaceutically acceptable buffering agent is a phosphate buffer, citrate buffer, tris buffer, histidine buffer or acetate buffer.

In some embodiments, a pharmaceutically acceptable buffering agent is sodium phosphate, dibasic. In some embodiments, a pharmaceutically acceptable buffering agent is sodium phosphate, monobasic. In some embodiments, a pharmaceutically acceptable buffering agent is a mixture of sodium phosphate, dibasic, and sodium phosphate, monobasic. In some embodiments, an ophthalmic solution of the invention comprises about 0.083% w/v sodium phosphate, dibasic, and about 0.017% w/v sodium phosphate, monobasic.

In some embodiments, the ophthalmic solution of the invention is at an approximately neutral pH. In some embodiments, an ophthalmic solution of the invention is at a pH of 6.5 to 8. In some embodiments, an ophthalmic solution of the invention is at a pH of 6.9 to 7.7. In some embodiments, an ophthalmic solution of the invention is at a pH of 7.1 to 7.5. In some embodiments, an ophthalmic solution of the invention is at a pH of about 7.3.

Pharmaceutically acceptable acids and/or bases may be used in the ophthalmic solution to adjust pH. In some embodiments, an ophthalmic solution of the invention comprises a pharmaceutically acceptable acid. In some embodiments, an ophthalmic solution of the invention comprises a pharmaceutically acceptable base. In some embodiments, an ophthalmic solution of the invention comprises a pharmaceutically acceptable acid and base. In some embodiments, a pharmaceutically acceptable acid is hydrochloric acid. In some embodiments, pharmaceutically acceptable base is sodium hydroxide.

In some embodiments, an ophthalmic solution of the invention comprises a tonicity agent. In some embodiments, a tonicity agent is selected from the group consisting of dextrose, potassium chloride, propylene glycol, and sodium chloride. In some embodiments, an ophthalmic solution of the invention comprises a tonicity agent at a concentration of less than about 0.5% w/v. In some embodiments, an ophthalmic solution of the invention comprises a tonicity agent at a concentration of about 0.45%, 0.4%, 0.35%, 0.3%, 0.25%, 0.2%, 0.15%, or 0.1% w/v. In some embodiments, a tonicity agent is sodium chloride.

In some embodiments, the ophthalmic solution comprises reproxalap at the specified concentrations, cyclodextrin, phosphate, and sodium chloride. In some embodiments. In some embodiments, the ophthalmic solution comprises reproxalap at the specified concentrations herein (e.g., 0.1% w/v, 0.25% w/v, 0.5% w/v, etc.), 5 to 9% w/v cyclodextrin (e.g., sulfobutylether-β-cyclodextrin or hydroxypropyl-β-cyclodextrin); 0.07% to 0.09% w/v sodium phosphate (dibasic), 0.015% to 0.19% w/v sodium phosphate (monobasic), and 0.2 to 0.3% w/v sodium chloride. In some embodiments, the ophthalmic solution comprises reproxalap at the specified concentrations herein (e.g., 0.1% w/v, 0.25% w/v, 0.5% w/v, etc.), about 7% w/v cyclodextrin (e.g., sulfobutylether-β cyclodextrin or hydroxypropyl-β-cyclodextrin); 0.07% to 0.09% w/v sodium phosphate (dibasic), 0.015% to 0.019% w/v sodium phosphate (monobasic), and 0.2 to 0.3% w/v sodium chloride. In some embodiments, the ophthalmic solution is adjusted to an appropriate pH with sodium hydroxide or HCL.

In some embodiments, the ophthalmic solution comprises the following (0.5% Reproxalap Ophthalmic Solution A):

| Component | Amount (% w/v) | Grade |
|---|---|---|
| ADX-102 (reproxalap) | 0.5% | GMP |
| Sulfobutylether-beta-cyclodextrin | 9.5% | USP |
| (SBECD) | | |
| Sodium phosphate, dibasic, anhydrous | 0.083% | USP |
| Sodium phosphate, monobasic, | 0.017% | USP |
| monohydrate | | |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.5% Reproxalap Ophthalmic Solution B)

| Component | Amount (% w/v) | Grade |
|---|---|---|
| ADX-102 (reproxalap) | 0.5% | GMP |
| Sulfobutylether-beta-cyclodextrin | 9.5% | USP |
| (SBECD) | | |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.250% Reproxalap Ophthalmic Solution A)

| Component | Amount (% w/v) | Grade |
|---|---|---|
| ADX-102 (reproxalap) | 0.25% | GMP |
| Sulfobutylether-beta-cyclodextrin | 7.0% | USP |
| (SBECD) | | |
| Sodium phosphate, dibasic, anhydrous | 0.083% | USP |
| Sodium phosphate, monobasic, | 0.017% | USP |
| monohydrate | | |
| Sodium chloride | 0.24% | USP |
| Sodium chloride | Tonicity adjustment | USP |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.25% Reproxalap Ophthalmic Solution B)

| Component | Amount (% w/v) | Grade |
|---|---|---|
| ADX-102 (reproxalap) | 0.25% | GMP |
| Sulfobutylether-beta-cyclodextrin | 7.0% | USP |
| (SBECD) | | |
| Sodium chloride | Tonicity adjustment | USP |
| Sodium hydroxide or Hydrochloric acid | pH adjustment | USP/NF |
| Sterile Water for Injection (WFI) | Dilute to volume | USP |

It is to be understood that variations of the ophthalmic solutions within the scope of the disclosure may be prepared given the guidance provided herein.

### 4. Methods of Treatment

In one aspect, the present invention provides a method for treating dry eye disease in a subject, comprising topically administering to an eye of a subject in need thereof a therapeutically effective amount of an ophthalmic solution of the invention. In some embodiments, the concentration of reproxalap in the ophthalmic solution used in the method is as described above.

In some embodiments, an ophthalmic solution of the invention can be administered at different frequencies suitable for effectively treating dry eye disease, for example, without causing severe or intolerable adverse effects.

In some embodiments, the ophthalmic solution is administered prophylactically to prevent the development or delay the onset of dry eye disease or its symptoms. In some embodiments, a subject with a prior history of dry eye disease but who is not experiencing or exhibiting symptoms of dry eye disease is selected for prophylactic treatment.

In some embodiments, a method for prophylactic treatment, for example to prevent the development or delay the onset of dry eye disease, comprises topically administering to an eye of a subject in need thereof a prophylactically effective amount of an ophthalmic solution disclosed herein. Any of the formulations of reproxalap disclosed herein can be used for prophylactic treatment. In some embodiments, the ophthalmic solution is administered topically one one to six times a day. In some embodiments, a method for prophylactic treatment of dry eye disease comprises topically administering an ophthalmic solution disclosed herein six times a day. In some embodiments, a method for prophylactic treatment of dry eye disease comprises topically administering an ophthalmic solution disclosed herein five times a day. In some embodiments, a method for prophylactic treatment of dry eye disease comprises topically administering an ophthalmic solution disclosed herein four times a day (QID). In some embodiments, a method for prophylactic treatment of dry eye disease comprises topically administering an ophthalmic solution disclosed herein three times a day (TID). In some embodiments, a method for prophylactic treatment of dry eye disease comprises topically administering an ophthalmic solution disclosed herein two times a day (BID). In some embodiments, a method for prophylactic treatment of dry eye disease comprises topically administering an ophthalmic solution disclosed herein once a day (QD). In some embodiments, a method for prophylactic treatment of dry eye disease comprises topically administering an ophthalmic solution disclosed herein as needed (PRN).

In some embodiments for prophylactic treatment, the ophthalmic solution is administered up to six times a day followed by a lower frequency of administration. In some embodiments, the ophthalmic solution is administered six times a day, followed by administration four times a day, three times a day, two times a day, or once a day. In some embodiments for prophylactic treatment, the ophthalmic solution is administered up to six times a day followed by administration as needed. In some embodiments, the ophthalmic solution is administered four times a day, followed by a lower frequency of administration. In some embodiments, the ophthalmic solution is administered four times a day, followed by administration three times a day, two times a day, or once a day. In some embodiments for prophylactic treatment, the ophthalmic solution is administered four times a day followed by administration as needed. In some embodiments, the ophthalmic solution is administered four times a day (QID) followed by administration two times a day (BID).

In some embodiments for prophylactic treatment, the ophthalmic solution disclosed herein is administered to a subject in need thereof once every two days, once every three days, once every four days, once every five days, once every six days, or once every week. In some embodiments, the ophthalmic solution disclosed herein is administered to a subject in need thereof once every 2 weeks, once every 3 weeks, or once every month. Each day of treatment can be any of the number of treatments disclosed above. In each day of treatment, the ophthalmic solution is administered six times a day, five times a day, four times a day, three times a day, two times a day, or once per day.

In some embodiments, prophylactic treatment can be for at least 2 days, 3 days, 4 days, 5 days, 6 days, or a week. In some embodiments, prophylactic treatment can be for a period of two weeks, three weeks, 4 weeks (1 month), 2 months, 3 months, 4 months, 5 months, 6 months, 7 months, 8 months, 9 months, 10 months, 11 months or up to one year. In some embodiments, the prophylactic treatment is continuous and maintained for as long as needed. In some embodiments, the prophylactic treatment is continuous and maintained for to keep levels of RASP, particularly in tears, that are within levels present in normal eyes, e.g., subjects who are not suffering from dry eye disease or other ocular inflammatory disorders.

In some embodiments, the ophthalmic solution is administered as a pre-treatment (also referred to as a pre-treatment phase) prior to therapeutic treatment or prior to an expected or certain exposure to one or more conditions that initiate dry eye disease or its symptoms. In some embodiments, a subject for pre-treatment has a prior history of dry eye disease. In some embodiments of a pre-treatment phase, the ophthalmic solution disclosed herein is administered topically one to six times a day. In some embodiments, a method for pre-treatment prior to an expected exposure to conditions that initiate dry eye disease or its symptoms comprises topically administering an ophthalmic solution disclosed herein six times a day. In some embodiments in the pre-treatment phase, an ophthalmic solution disclosed herein is administered five times a day. In some embodiments in the pre-treatment phase, an ophthalmic solution disclosed is administered four times a day (QID). In some embodiments in the pre-treatment phase, an ophthalmic solution disclosed herein is administered three times a day (TID). In some embodiments in the pre-treatment phase, an ophthalmic solution disclosed herein is administered two times a day (BID). In some embodiments in the pre-treatment phase, an ophthalmic solution disclosed herein is administered once a day (QD). In a preferred embodiment, an ophthalmic solution is administered four times a day in the pre-treatment phase.

In some embodiments in a pre-treatment phase, an ophthalmic solution disclosed herein is administered up to six times a day followed by a lower frequency of administration. In some embodiments in the pre-treatment phase, an ophthalmic solution disclosed herein is administered six times a day, followed by administration four times a day, three times a day, two times a day, or once a day. In some embodiments in the pre-treatment phase, an ophthalmic solution disclosed herein is administered up to six times a day followed by administration as needed. In some embodiments in the pre-treatment phase, an ophthalmic solution is administered four times a day, followed by a lower frequency of administration. In some embodiments in the pre-treatment phase, an ophthalmic solution is administered four times a day, followed by administration three times a day, two times a day, or once a day. In some embodiments in the pre-treatment phase, the ophthalmic solution is administered four times a day followed by administration as needed. In some embodiments in the pre-treatment phase, the ophthalmic solution is administered four times a day (QID) followed by administration two times a day (BID).

In some embodiments of pre-treatment, an ophthalmic solution disclosed herein is administered 5 days or less, four day or less, three days or less, two days or less, one day or less, and/or immediately prior to an expected or certain exposure to one or more conditions that initiate dry eye disease or its symptoms. In some embodiments in a pre-treatment phase, an ophthalmic solution disclosed herein is administered 2 days or less before an expected or certain exposure to one or more conditions that initiate dry eye disease or its symptoms. In some embodiments in a pre-treatment phase, an ophthalmic solution disclosed herein is administered 1 day or less before an expected or certain exposure to one or more conditions that initiate dry eye disease or its symptoms. In some embodiments in a pre-treatment phase, an ophthalmic solution disclosed herein is administered immediately before an expected or certain exposure to one or more conditions that initiate dry eye disease or its symptoms. In some embodiments in a pre-treatment phase, an ophthalmic solution disclosed herein is administered about 30 min, about 25 min, about 20 min, about 15 min, about 10 min, about 5 min, or about 2 min before an expected or certain exposure to one or more conditions that initiate dry eye disease or its symptoms. In some embodiments of a pre-treatment phase, an ophthalmic solution disclosed herein is administered 2 days before, 1 day before, and immediately prior to to an expected or certain exposure to one or more conditions that initiate dry eye disease or its symptoms. In some embodiments of a pre-treatment phase, an ophthalmic solution disclosed herein is administered 1 day before and immediately prior to an expected or certain exposure to one or more conditions that initiate dry eye disease or its symptoms.

In some embodiments, an ophthalmic solution disclosed herein is administered to treat dry eye disease. In some embodiments, the ophthalmic solution disclosed herein is administered at the onset of dry eye disease or symptoms of dry eye disease. In some embodiments, the ophthalmic solution disclosed herein is administered following the pre-treatment phase discussed above, for example, after exposure to one or more conditions that initiate dry eye disease or its symptoms. In some embodiments, a method for treating dry eye disease comprises topically administering to a subject in need thereof a therapeutically effective amount of an ophthalmic solution disclosed herein. In some embodiments, an ophthalmic solution disclosed herein is topically administered one to six times a day. In some embodiments, an ophthalmic solution disclosed herein is administered six times a day. In some embodiments, an ophthalmic solution disclosed herein is administered five times a day. In some embodiments, an ophthalmic solution disclosed herein is administered four times a day (QID). In some embodiments, an ophthalmic solution disclosed herein is administered three times a day (TID). In some embodiments, an ophthalmic solution disclosed herein is administered two times a day (BID). In some embodiments, an ophthalmic solution disclosed herein is administered once a day (QD). In some embodiments for treating dry eye disease, an ophthalmic solution disclosed herein is administered as needed (PRN).

In some embodiments, a method of the invention comprises topically administering to an eye of a subject with dry eye disease a therapeutically effective amount of an ophthalmic solution of the invention six times a day, five times a day, four times a day (QID), three times a day (TID), two times a day (BID), or once a day (QD), followed by administration as needed (PRN).

In some embodiments, a method of the invention comprises topically administering an ophthalmic solution disclosed herein at various strengths (for example, at different reproxalap concentrations and different administration frequencies, as described herein).

In some embodiments, a method of the invention comprises topically administering an ophthalmic solution comprising about 0.25% w/v reproxalap, or a pharmaceutically acceptable salt thereof, four times a day, three times a day, or two times a day.

In some embodiments, a method of the invention comprises topically administering an ophthalmic solution comprising about 0.30% w/v reproxalap, or a pharmaceutically acceptable salt thereof, four times a day, three times a day, or two times a day.

In some embodiments, a method of the invention comprises topically administering an ophthalmic solution comprising about 0.35% w/v reproxalap, or a pharmaceutically acceptable salt thereof, four times a day, three times a day, or two times a day.

In some embodiments, a method of the invention comprises topically administering an ophthalmic solution comprising about 0.4% w/v reproxalap, or a pharmaceutically acceptable salt thereof, four times a day, three times a day, or two times a day.

In some embodiments, a method of the invention comprises topically administering an ophthalmic solution comprising about 0.45% w/v reproxalap, or a pharmaceutically acceptable salt thereof, four times a day, three times a day, or two times a day.

In some embodiments, a method of the invention comprises topically administering an ophthalmic solution comprising about 0.5% w/v reproxalap, or a pharmaceutically acceptable salt thereof, four times a day, three times a day, or two times a day.

In some embodiments, a method of the invention comprises topically administering an ophthalmic solution comprising 0.3% to 0.4% w/v reproxalap, or a pharmaceutically acceptable salt thereof, four times a day, three times a day, or two times a day.

In some embodiments, a method of the invention comprises topically administering an ophthalmic solution comprising 0.2% to 0.3% w/v reproxalap, or a pharmaceutically acceptable salt thereof, four times a day, three times a day, or two times a day.

In some embodiments, a method of the invention comprises topically administering an ophthalmic solution comprising 0.2% to 0.4% w/v reproxalap, or a pharmaceutically acceptable salt thereof, four times a day, three times a day, or two times a day.

In some embodiments, a method of the invention comprises two or more phases, wherein an ophthalmic solution of the invention is topically administering at different strengths in different phases. In some embodiments, a method of the invention comprises an initiation phase and a maintenance phase, wherein the ophthalmic solution is topically administered at a higher strength in the initiation phase than in the maintenance phase. In some embodiments, a treatment cycle of a method of the invention comprising multiple phases, including an exacerbation phase during which signs and/or symptoms become worse.

In some embodiments, the method of the invention comprises two or more phases, wherein an ophthalmic solution of the invention is topically administering at different strengths in different phases. In some embodiments, a method of the invention comprises an initiation phase, wherein the ophthalmic solution is topically administered at a high strength in the initiation phase, at a low strength in the maintenance phase, and at a high strength during an exacerbation of disease signs and/or symptoms.

In some embodiments, an ophthalmic solution administered in an initiation phase comprises a higher concentration of reproxalap, or a pharmaceutically acceptable salt thereof, than an ophthalmic solution administered in a maintenance phase. In some embodiments, the ophthalmic solution administered in an initiation phase or an exacerbation phase and the ophthalmic solution administered in a maintenance phase, comprises reproxalap, or a pharmaceutically acceptable salt, at a concentration selected from the group consisting of about 0.5% w/v, 0.45% w/v, 0.4% w/v, 0.35% w/v, 0.3% w/v, 0.25% w/v, 0.2% w/v, 0.15% w/v, and 0.1% w/v.

In some embodiments, an ophthalmic solution of about 0.5% w/v reproxalap is administered in an initiation phase or exacerbation phase, and less than 0.5% w/v reproxalap administered in a maintenance phase. In some embodiments, an ophthalmic solution of about 0.4% w/v, 0.35% w/v, 0.3% w/v, 0.25% w/v, 0.2% w/v, 0.15% w/v or 0.1% w/v reproxalap is administered in the maintenance phase.

In some embodiments, an ophthalmic solution of about 0.5% w/v to about 0.4% reproxalap is administered in an initiation phase or exacerbation phase, and less than 0.4% w/v reproxalap administered in a maintenance phase. In some embodiments, an ophthalmic solution of about 0.35% w/v, 0.3% w/v, 0.25% w/v, 0.2% w/v, 0.15% w/v or 0.1% w/v reproxalap is administered in the maintenance phase.

In some embodiments, an ophthalmic solution of about 0.5% w/v to about 0.3% reproxalap is administered in an initiation phase or exacerbation phase, and less than 0.3% w/v reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of about 0.4% w/v to about 0.3% reproxalap is administered in an initiation phase or exacerbation phase, and less than 0.3% w/v reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of about 0.3% w/v to about 0.2% reproxalap (e.g., 0.3%, 0.25%, or 0.2% w/v) is administered in an initiation phase or exacerbation phase, and 0.25% w/v or less reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of the invention is topically administered more frequently per day in an initiation phase and an exacerbation phase than in a maintenance phase. In some embodiments, an ophthalmic solution of the invention is topically administered five times a day in an initiation phase, followed by four, three, two, or one times a day in a maintenance phase. In some embodiments, an ophthalmic solution of the invention is topically administering four times a day in an initiation phase or exacerbation phase, followed by three, two, or one times a day in a maintenance phase. In some embodiments, an ophthalmic solution of the invention is topically administering three times a day in an initiation phase or exacerbation phase, followed by two or one times a day in a maintenance phase. In some embodiments, an ophthalmic solution of the invention is topically administering two times a day in an initiation phase or exacerbation phase, followed by once daily in a maintenance phase.

In some embodiments, an ophthalmic solution administered in an initiation phase or exacerbation phase is at a higher reproxalap concentration and higher administration frequency than an ophthalmic solution administered in a maintenance phase.

In some embodiments, the present invention provides a method for treating dry eye disease in a subject, comprising topically administering to the subject an ophthalmic solution comprising about 0.4% w/v reproxalap, or a pharmaceutically acceptable salt thereof, and a pharmaceutically acceptable excipient, wherein the ophthalmic solution is administered at a higher strength in an initiation phase or exacerbation phase followed by a lower strength in a maintenance phase, wherein each of the initiation phase, exacerbation phase, and maintenance phase is as described herein.

In some embodiments, a multiple phase treatment cycle can include an initiation phase or exacerbation phase of up to 12 weeks with an ophthalmic solution comprising about 0.5%, 0.4% or 0.35% w/v (e.g., 0.5% to 0.35% w/v) reproxalap, or a pharmaceutically acceptable salt thereof, is up to 12 weeks, followed by a maintenance phase. In some embodiments, an ophthalmic solution comprising about 0.5%, 0.4% or 0.35% w/v (e.g., 0.5% to 0.35% w/v) reproxalap, or a pharmaceutically acceptable salt thereof, is administered four times a day in an initiation phase or exacerbation phase followed by three, two, or one times a day in the maintenance phase. In some embodiments, an ophthalmic solution comprising about 0.5%, 0.4% or 0.35% w/v (e.g., 0.5% to 0.35% w/v) reproxalap, or a pharmaceutically acceptable salt thereof, is administered three times a day in an initiation phase or exacerbation phase followed by two or one times a day in the maintenance phase.

In some embodiments, an ophthalmic solution comprising about 0.4%, 0.35% or 0.3% w/v (e.g., 0.4% to 0.3% w/v) reproxalap, or a pharmaceutically acceptable salt thereof, is administered four times a day in an initiation phase or exacerbation phase followed by three, two, or one times a day in the maintenance phase. In some embodiments, an ophthalmic solution comprising about 0.4%, 0.35% or 0.3% w/v (e.g., 0.4% to 0.3% w/v) reproxalap, or a pharmaceutically acceptable salt thereof, is administered three times a day in an initiation phase or exacerbation phase followed by two or one times a day in the maintenance phase.

In some embodiments, an ophthalmic solution comprising about 0.3%, 0.25% or 0.2% w/v (e.g., 0.3% to 0.2% w/v) reproxalap, or a pharmaceutically acceptable salt thereof, is administered four times a day in an initiation phase or exacerbation phase followed by three, two, or one times a day in the maintenance phase. In some embodiments, an ophthalmic solution comprising about 0.3%, 0.25% or 0.2% w/v (e.g., 0.3% to 0.2% w/v) reproxalap, or a pharmaceutically acceptable salt thereof, is administered three times a day in an initiation phase or exacerbation phase followed by two or one times a day in the maintenance phase. In some embodiments, an ophthalmic solution comprising about 0.3%, 0.25% or 0.2% w/v (e.g., 0.3% to 0.2% w/v) reproxalap, or a pharmaceutically acceptable salt thereof, is administered four times a day (QID) followed by administration two times a day (BID).

In some embodiments, the present invention provides a method for treating dry eye disease in a subject, comprising topically administering to the subject an ophthalmic solution comprising 0.35% to 0.45% w/v reproxalap, or a pharmaceutically acceptable salt thereof, and a pharmaceutically acceptable excipient, wherein the ophthalmic solution is administered at a higher strength in an initiation phase or exacerbation phase followed by a lower strength in a maintenance phase, wherein each of the initiation phase, exacerbation phase and maintenance phase is as described herein. In some embodiments, a multiple phase treatment cycle of an ophthalmic solution comprising 0.35% to 0.45% w/v reproxalap, or a pharmaceutically acceptable salt thereof, is up to 12 weeks. In some embodiments, an ophthalmic solution comprising 0.35% to 0.45% w/v reproxalap, or a pharmaceutically acceptable salt thereof, is administered four times a day in an initiation phase or exacerbation phase followed by three, two, or one times a day in maintenance phase. In some embodiments, an ophthalmic solution comprising 0.35%-0.45% w/v reproxalap, or a pharmaceutically acceptable salt thereof, is administered three times a day in an initiation phase or exacerbation phase followed by two or one times a day in maintenance phase.

In some embodiments, an ophthalmic solution is administered QID for about 10 to 14 weeks, preferably about 12 weeks. In some embodiments, an ophthalmic solution is administration QID for about 2 to 6 weeks, preferably about 4 weeks followed by administration BID for about 6 to 10 weeks, preferably about 8 weeks. In some embodiments, the ophthalmic solution for the foregoing treatment regimen is 0.25% w/v reproxalap, or a pharmaceutically acceptable salt thereof, and about 7% w/v SBECD.

In some embodiments, an ophthalmic solution is administered QID for about 2 to 6 weeks, preferably about 4 weeks, followed by administration BID for about 6 to 10 weeks, preferably about 8 weeks. In some embodiments, the ophthalmic solution for the foregoing treatment regimen is 0.25% w/v reproxalap, or a pharmaceutically acceptable salt thereof, and about 11% w/v SBECD.

In some embodiments, the present invention provides a method for treating certain subjects with dry eye disease. In some embodiments, a subject with dry eye disease is 18 years or older. In some embodiments, a subject with dry eye disease has a history of dry eye for at least six months prior to receiving the treatment of the invention. In some embodiments, a subject with dry eye disease has a history of use or desire to use eye drops for dry eye symptoms within six months prior to receiving the treatment of the invention.

In some embodiments, the present invention provides a method for treating a subject with dry eye disease, in particular moderate-to-severe dry-eye disease, comprising identifying subjects satisfying one or more of the following criteria for at least one eye, prior to receiving the treatment of the invention (for example, a screening performed at about one and/or two weeks before receiving the treatment):
having a Schirmer's Test score of ≤10 mm and ≥1 mm;
having a tear film break-up time (TFBUT©)≤5 seconds;
having a corneal fluorescein staining score of ≥2 in at least one region (e.g., inferior, superior, or central);
having a sum corneal fluorescein staining score of ≥4 based on the sum of the inferior, superior, and central regions; and
having a total Lissamine green conjunctival score of 2 based on the sum of the temporal and nasal regions.

In some embodiments, a subject with dry eye disease is not a female patient who is pregnant, nursing, or planning a pregnancy. In some embodiments, a subject with dry eye disease has not previously used reproxalap ophthalmic solution.

In some embodiments, the present invention provides a method for treating a subject with dry eye disease comprising a screening to exclude subjects having one or more of the following conditions for at least one eye, prior to receiving the treatment of the invention:
having any clinically significant slit lamp findings that may include active blepharitis, meibomian gland dysfunction (MGD), lid margin inflammation, or active ocular allergies that may require therapeutic treatment;
having an ongoing ocular infection (bacterial, viral, or fungal), or active ocular inflammation;
having previously had laser-assisted in situ keratomileusis (LASIK) surgery within the last 12 months;
having any planned ocular and/or lid surgeries over the study period or any ocular surgery within six months; and
having a known allergy and/or sensitivity to an ophthalmic solution of the invention or its components.

In some embodiments, administration of an ophthalmic solution disclosed herein can provide immediate improvements in treating dry eye disease. In some embodiments, an ophthalmic solution is administered to an eye in need thereof on or immediately after onset of symptoms of dry eye disease. In some embodiments, reproxalap ophthalmic solution is administered within minutes of onset of symptoms of dry eye disease. In some embodiments, an ophthalmic solution disclosed herein is administered within 5 min, 10 min, 15 min, 20 min, 25 min, 30 min, 35 min, 40 min, 45 min, 50 min or 60 min of onset of symptoms of dry eye disease. In some embodiments, the ophthalmic solution is administered within minutes of onset of symptoms of dry eye disease followed by administration at about 45 min, 50 min, 55 min, or 60 min of onset of symptoms of dry eye disease

In some embodiments, administration of an ophthalmic solution disclosed herein can achieve an early onset of effect in subjects with dry eye disease. As used herein, an “early onset effect” refers to early efficacy (e.g., within 1 to 2 weeks of initiation of treatment—in initiation or exacerbation phase) in ameliorating symptoms of dry eye disease. In some embodiments, the “early onset effect” is for the same dose and frequency of administration in the initiation or exacerbation phase. Accordingly, in some embodiments, the present invention provides a method for treating a subject with dry eye disease comprising topically administering to the subject an ophthalmic solution of the invention, wherein the ophthalmic solution is administered at a dose strength which can achieve an early onset profile. In some embodiments, an early onset profile comprises early onset of effect for symptoms (e.g., ocular discomfort including dryness, itchiness, tearing, burning, stinging, grittiness, cloudy vision, sensitivity to environment, stringy ocular secretion). In some embodiments, an early onset profile comprises early onset of effect for signs (e.g., ocular vital staining, tear film break-up time, tear osmolarity, tear volume).

In some embodiments, a dose strength which can achieve an early onset of effect comprises topically administering an ophthalmic solution comprising reproxalap, or a pharmaceutically acceptable salt thereof, at a concentration as described herein. In some embodiments, a dose strength which can achieve an early onset of effect comprises topically administering an ophthalmic solution comprising reproxalap, or a pharmaceutically acceptable salt thereof, at a frequency at described herein. In some embodiments, a dose strength which can achieve an early onset of effect comprises topically administering an ophthalmic solution comprising reproxalap, or a pharmaceutically acceptable salt thereof, at a concentration and a frequency at described herein.

In some embodiments, a method of the invention can achieve an onset of effect in about two weeks. At different dose strengths (for example, different concentration and administering frequency), a method of the invention can achieve an onset in fewer than about two weeks. For example, in some embodiments, a method of the invention can achieve an onset in about 14, 13, 12, 11, ten, nine, or eight days. At a certain dose strength, a method of the invention can achieve an onset in about one week or less. In some embodiments, a method of the invention can achieve an onset in about seven, six, five, four, three, two, or one days.

In some embodiments, the present invention provides a method for treating dry eye disease in a subject, comprising topically administering to the subject an ophthalmic solution comprising about 0.5% w/v reproxalap, or a pharmaceutically acceptable salt thereof, and a pharmaceutically acceptable excipient, wherein the ophthalmic solution is administered three, two, or one times a day. In some embodiments, an ophthalmic solution comprising about 0.5% w/v reproxalap, or a pharmaceutically acceptable salt thereof, is administered three times a day. In some embodiments, an ophthalmic solution comprising about 0.5% w/v reproxalap, or a pharmaceutically acceptable salt thereof, is administered two times a day. In some embodiments, an ophthalmic solution comprising about 0.5% w/v reproxalap, or a pharmaceutically acceptable salt thereof, is administered once daily.

In some embodiments, the present invention provides a method for treating dry eye disease in a subject, comprising topically administering to the subject an ophthalmic solution comprising about 0.5% w/v reproxalap, or a pharmaceutically acceptable salt thereof, and a pharmaceutically acceptable excipient, wherein the ophthalmic solution is administered at a higher strength in an initiation phase or exacerbation phase, followed by a lower strength in a maintenance phase, wherein each of the initiation phase, exacerbation phase, and maintenance phase is as described herein. In some embodiments, an ophthalmic solution comprising about 0.5% w/v reproxalap, or a pharmaceutically acceptable salt thereof, is administered four times a day in an initiation phase or exacerbation phase followed by three, two, or one times a day in a maintenance phase. In some embodiments, an ophthalmic solution comprising about 0.5% w/v reproxalap, or a pharmaceutically acceptable salt thereof, is administered three times a day in an initiation phase or exacerbation phase followed by two or one times a day in a maintenance phase. In some embodiments, an ophthalmic solution comprising about 0.5% w/v reproxalap, or a pharmaceutically acceptable salt thereof, is administered two times a day in an initiation phase followed by one time a day in a maintenance phase. In some embodiments, an ophthalmic solution comprising about 0.5% w/v reproxalap, or a pharmaceutically acceptable salt thereof, is topically administered in an initiation phase or exacerbation phase, followed by topical administration of an ophthalmic solution comprising less than about 0.5% w/v reproxalap, or a pharmaceutically acceptable salt thereof, in a maintenance phase, wherein the administration frequency of each ophthalmic solution is selected from those as described above.

In some embodiments, a provided method provides an improvement in tear quantity and/or quality in the eye of the subject. In some embodiments, the improvement is measured using Schirmer's Test. In some embodiments the improvement is an improvement in tear quantity. In some embodiments, the improvement is an improvement in tear quality. Tear quality may be measured using various methods known in the art. In some embodiments, tear quality is measured using non-invasive tear break up time (NITBUT). This is the measurement, in seconds, of the time that elapses between the last complete blink and the appearance of the first discontinuity in the tear film. The one-position keratometer is the most commonly available instrument which can be employed to measure NITBUT in clinical practice. For measurement, the practitioner observes the keratometer mires and records the time taken from a complete blink until the mires begin to distort and/or break up. One consideration which could affect measurement is that the keratometer is limited to assessing a small area, only providing information on tear break up in the central cornea. In some embodiments, tear quality is measured using a Placido disc topographer, which uses white illumination and Placido discs to visualize the tear film. These instruments allow visibility of the majority of the corneal surface and can automatically detect and record the time of first break up. In some embodiments, tear quality is measured using a slit lamp. The lipid layer can be viewed by utilizing the technique of specular reflection on a slit lamp. Viewing the first Purkinje image with a narrow slit beam, lipid interference patterns can be observed. As a general rule, the brighter the colored fringes appear, the thicker the lipid layer, whereas a dull, grey appearance may indicate a thinner layer. While a useful technique, the observer is limited to viewing a small area at a time, and the practitioner should be cautious that the heat of the slit lamp does not produce artificial drying. In some embodiments, tear quality is measured using Lipid layer interferometry. This technique can also be employed to assess the lipid layer thickness and many of the dry eye diagnostic devices include this feature. This is a more advanced examination method, giving practitioners an accurate and quantitative measurement of this delicate layer, with a healthy lipid layer thought to be approximately 40 nm thick. Considering the importance of the lipid layer in maintaining tear film stability, interferometry can be a useful tool for practitioners wanting to examine the lipid layer in greater detail. Traditionally, tear break-up time has been measured by staining the “transparent” tears with fluorescein to assist with observing and viewing the tear film under cobalt blue light. Additional use of a yellow “Wratten” filter further improves observation of fluorescence. The stain is usually applied by wetting a fluorescein-impregnated strip with saline, then shaking off any excess liquid and gently touching the conjunctiva with the strip tip. Touching the eye with the paper strip will induce a degree of reflex tearing and instilling too much fluorescein may swamp the normal 8 μl tear film, destabilizing it. Furthermore, the addition of fluorescein to the tear film alters the physical interactions between its layers, which reduces the surface tension and, hence, affects the break-up time value. It should be noted that while this technique is invasive, it is still widely used as a method of tear film assessment in practice. Studies have shown that when care is taken to instill a minimal amount of fluorescein that results are comparable with non-invasive techniques. Using this technique, a value of less than 10 seconds is typically considered abnormal.

In some embodiments, the QID administration in the initiation phase produces an improvement in tear quantity and/or quality in the eye of the subject. In some embodiments, the improvement is measured using Schirmer's Test. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

In some embodiments, a provided method provides an improvement in ocular redness.

In some embodiments, the QID administration in the initiation phase produces an improvement in ocular redness. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

In some embodiments, a provided method provides an improvement in ocular dryness.

In some embodiments, the QID administration in the initiation phase produces an improvement in ocular dryness. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

In some embodiments, a provided method provides an improvement in ocular itch.

In some embodiments, the QID administration in the initiation phase produces an improvement in ocular itch. In some embodiments, the improvement is greater than that observed vs. administration once a day, BID, or TID under otherwise similar conditions. In some embodiments, the improvement is statistically significant (with a p value equal to or less than 0.05, e.g., between 0.05 and 0.0001) across a group of subjects, for example, as described in the Exemplification section below.

### EXEMPLIFICATION

The following examples are intended to illustrate the invention and are not to be construed as being limitations thereon. Reproxalap can be synthesized as reported previously, for example, in WO 2006/127945, the entire content of which is incorporated herein by reference.

### Abbreviations

CAE®: controlled adverse environment
GMP: Good Manufacturing Practice
ICH: International Council for Harmonization of Technical Requirements for
Pharmaceuticals for Human Use
OD: right eye
OS: left eye
OU: both eyes
PRN: as needed
QD: once daily
QID: Four times daily
QS: as much as will suffice

### Example 1. A Multi-Center, Phase 2b, Randomized, Double-Masked, Parallel-Group, Vehicle-Controlled, Clinical Study to Assess the Safety and Efficacy of Reproxalap Ophthalmic Solution (0.25% and 0.1%) Compared to Vehicle in Subjects with Dry Eye Disease

Objectives:
To evaluate the efficacy of Reproxalap Ophthalmic Solutions (0.25% and 0.1%) on baseline to weeks 2, 4, 8, and 12 change scores for sign and symptom endpoints of dry eye disease.
To evaluate effect sizes for efficacy endpoints of Reproxalap Ophthalmic Solutions (0.25% and 0.1%) vs vehicle for the treatment of the signs and symptoms of dry eye disease to confirm the endpoint selection and sample size for Phase 3 studies.
To evaluate the safety and tolerability of Reproxalap Ophthalmic Solutions (0.25% and 0.1%) to vehicle for the treatment of the signs and symptoms of dry eye disease.

Investigational Product:
1) Reproxalap Ophthalmic Solution (0.25%)
2) Reproxalap Ophthalmic Solution (0.1%)
3) Vehicle Ophthalmic Solution

In the Phase 2b study, reproxalap was formulated as an ophthalmic solution as described in the specification.

Duration: A subject's participation was estimated to be approximately 14 weeks (98 days).

Dosage/Dose Regimen/Instillation/Application/Use: Screening: Between Visits 1 and 2, all subjects received 14 consecutive days (±2) of Run-in (vehicle) ocular drops self-administered QID in both eyes.

Treatment: During the 12-week (84±3 days) treatment period, Reproxalap Ophthalmic Solution at concentrations of 0.1%, 0.25%, or vehicle ophthalmic solution was administered QID by bilateral topical ocular dosing. Subjects were randomized to one of three treatment groups (1:1:1) to receive study drug after the Post-CAE® assessments at Visit 2.

Summary of Visit Schedule: Six visits over the course of approximately 14 weeks
Visit 1=Day −14±2, CAE® Screening
Visit 2=Day 1, CAE® Confirmation/Baseline
Visit 3=Day 15±2, 2-Week Follow-Up
Visit 4=Day 29±2, 4-Week Follow-Up
Visit 5=Day 57±3, 8-Week Follow-Up
Visit 6=Day 85±3, 12-Week CAE® Follow-Up & Study Exit

Condition/Disease: Dry Eye Disease (DED)

Inclusion Criteria: Subjects for treatment were based on the following criteria:
1 Been at least 18 years of age of either gender and any race;
2 Provide written informed consent and sign the Health Information Portability and Accountability Act (HIPAA) form;
3 Had a reported history of dry eye for at least six months prior to Visit 1;
4 Had a history of use or desire to use eye drops for dry eye symptoms within six months of Visit 1;
5 Reported a score of ≥2 on the Ora Calibra® Ocular Discomfort & 4-Symptom Questionnaire in at least one symptom at Visit 1 and Visit 2 Pre-CAE®;
6 Had a Schirmer's Test score of ≤10 mm and ≥1 mm at Visit 1 and Visit 2;
7 Had a tear film break-up time (TFBUT©)≤5 seconds at Visit 1 and Visit 2 Pre-CAE®;
8 Had a corneal fluorescein staining score of ≥2 in at least one region (e.g., inferior, superior, or central) at Visit 1 and Visit 2 Pre-CAE®;
9 Have a sum corneal fluorescein staining score of ≥4, based on the sum of the inferior, superior, and central regions, at Visit 1 and Visit 2 Pre-CAE®;
10 Had a total Lissamine green conjunctival score of ≥2, based on the sum of the temporal and nasal regions at Visit 1 and Visit 2 Pre-CAE®;
11 Demonstrated a response to the CAE® at Visits 1 and 2 as defined by:
   A. Having at least a ≥1 point increase in fluorescein staining in the inferior region in at least one eye following CAE® exposure;
   B. Reporting an Ocular Discomfort score ≥3 at two or more consecutive time points in at least one eye during CAE® exposure (if a subject had an Ocular Discomfort rating of 3 at time=0 for an eye, s/he must have reported an Ocular Discomfort rating of 4 for two consecutive measurements for that eye). Note: a subject could not have an Ocular Discomfort score of 4 at time=0);
12 Had at least one eye, the same eye, satisfy all criteria for 6, 7, 8, 9, 10, and 11 above.

Exclusion Criteria: Subject were excluded based on the following criteria:
1 Had any clinically significant slit lamp findings at Visit 1 that may have included active blepharitis, meibomian gland dysfunction (MGD), lid margin inflammation, or active ocular allergies that require therapeutic treatment, and/or in the opinion of the investigator, might have interfered with study parameters;
2 Been diagnosed with an ongoing ocular infection (bacterial, viral, or fungal), or active ocular inflammation at Visit 1;
3 Worn contact lenses within seven days of Visit 1 or anticipate using contact lenses during the study;
4 Used any eye drops within 2 hours of Visit 1;
5 Had laser-assisted in situ keratomileusis (LASIK) surgery within the last 12 months;
6 Used cyclosporine 0.05% or lifitegrast 5.0% ophthalmic solution within 90 days of Visit 1;
7 Had any planned ocular and/or lid surgeries over the study period or any ocular surgery within 6 months of Visit 1;
8 Been using or anticipated using temporary punctal plugs during the study that had not been stable within 30 days of Visit 1;
9 Been currently taking any topical ophthalmic prescription (including medications for glaucoma) or over-the-counter (OTC) solutions, artificial tears, gels or scrubs, and cannot discontinue these medications for the duration of the trial (excluding medications allowed for the conduct of the study);
10 Had corrected visual acuity greater than or equal to logarithm of the minimum angle of resolution (log MAR)+0.7 as assessed by Early Treatment of Diabetic Retinopathy Study (ETDRS) scale in both eyes at Visit 1;
11 Been a woman who is pregnant, nursing, or planning a pregnancy;
12 Been unwilling to submit a urine pregnancy test at Visit 1 and Visit 6 (or early termination visit) if of childbearing potential. Non-childbearing potential was defined as a woman who is permanently sterilized (e.g., has had a hysterectomy or tubal ligation), or was postmenopausal (without menses for 12 consecutive months);
13 Been a man or woman of childbearing potential who was not using an acceptable means of birth control; acceptable methods of contraception include: hormonal—oral, implantable, injectable, or transdermal contraceptives; mechanical—spermicide in conjunction with a barrier such as a diaphragm or condom; intrauterine device (IUD); or surgical sterilization of partner. For non-sexually active males or females, abstinence may have been regarded as an adequate method of birth control; however, if the subject became sexually active during the study, he/she must have agreed to use adequate birth control as defined above for the remainder of the study;
14 Had a known allergy and/or sensitivity to the test article or its components;
15 Had a condition or be in a situation which the investigator feels may have put the subject at significant risk, confounded the study results, or interfered significantly with the subject's participation in the study;
16 Been currently enrolled in an investigational drug or device study or have used an investigational drug or device within 30 days of Visit 1;
17 Previously used reproxalap ophthalmic solution;
18 Been currently using any medication known to cause ocular drying that was not used on a stable dosing regimen for at least 30 days prior to Visit 1;
19 Been unable or unwilling to follow instructions, including participation in all study assessments and visits.

The following efficacy measures and endpoints were used in the study:
Lissamine green staining (Ora Calibra® scale); regions: inferior, superior, central, temporal, nasal, corneal sum, conjunctival sum, and total eye score)
Fluorescein staining (Ora Calibra® scale); regions: central, superior, inferior, temporal, nasal, corneal sum, conjunctival sum, and total eye score)
Tear film break-up time
Unanesthetized Schirmer's Test
Ora Calibra® Ocular Discomfort Scale
Ora Calibra® Ocular Discomfort & 4-Symptom Questionnaire
Ocular Surface Disease Index (OSDI)©
SANDE questionnaire
Tear Osmolarity

Safety Measures:
Visual acuity
Slit-lamp evaluation
Adverse event query
Intraocular Pressure (IOP)
Dilated fundoscopy

General Statistical Methods and Types of Analyses

Sample Size: The study sample size of 100 per group was selected based on prior Phase 2 and 3 clinical trial results using the DED Hybrid CAE® study design with other development programs and the effect size seen in Phase 2a with reproxalap on change from baseline after four weeks of treatment. This sample size was deemed sufficient to assess the effect size on the DED sign and symptom endpoints with reproxalap vs vehicle, to confirm the endpoint selection and sample size needed for Phase 3 studies with reproxalap. A sample size of 100 per group provided 90% power at α=0.05 to detect an effect size of 0.26 for inferior Lissamine green staining (Ora Calibra® scale), assuming a common standard deviation of 0.56 and an effect size of 0.44 for ocular discomfort assessed with the Ora Calibra® Ocular Discomfort Scale assuming a common standard deviation of 0.97.

Efficacy Analysis
Evaluated baseline to weeks 2, 4, 8 and 12 change scores with reproxalap on DED sign and symptom endpoints (both pre-CAE and CAE® endpoints). Each endpoint was analyzed at a two-sided alpha level of 0.05, and the overall type I error was not controlled for in this investigative study.
Evaluated effect size of baseline to weeks 2, 4, 8 and 12 change scores of reproxalap vs vehicle on DED sign and symptom endpoints (both pre-CAE and CAE® endpoints) to confirm the endpoint selection for primary outcome parameters and sample size for Phase 3 studies with reproxalap.
Sub-group analyses on effect size of baseline to weeks 2, 4, 8 and 12 change scores of reproxalap vs vehicle on DED sign and symptom endpoints (both pre-CAL and CAE® endpoints) [Subgroups were prospectively detailed in the Statistical Analysis Plan (SAP)].

TABLE 1
Summary of Subject Disposition

| | Reproxalap (0.1%) | | Reproxalap (0.25%) | | Vehicle | | All Subjects | |
|---|---|---|---|---|---|---|---|---|
| | N = 100 | | N = 100 | | N= 100 | | N = 300 | |
| Intent-to-Treat Population | 100 | (100.0%) | 100 | (100.0%) | 100 | (100.0%) | 300 | (100.0%) |
| Per Protocol Population | 97 | (97.0%) | 86 | (86.0%) | 98 | (98.0%) | 281 | (93.7%) |
| Safety Population | 100 | (100.0%) | 100 | (100.0%) | 100 | (100.0%) | 300 | (100.0%) |
| Study Completion | | | | | | | | |
| Completed | 97 | (97.0%) | 88 | (88.0%) | 99 | (99.0%) | 284 | (94.7%) |
| Discontinued | 3 | (3.0%) | 12 | (12.0%) | 1 | (1.0%) | 16 | (5.3%) |
| Reason for Study Withdrawal | | | | | | | | |

| | | | | | | | |
|---|---|---|---|---|---|---|---|
| Adverse Events | 2 | (2.0%) | 10 | (10.0%) | 0 | 12 | (4.0%) |

| | | | | | | |
|---|---|---|---|---|---|---|
| Administrative Reasons | 1 | (1.0%) | 0 | 0 | 1 | (0.3%) |

| | | | | | | | |
|---|---|---|---|---|---|---|---|
| Withdrawal by Subject | 0 | 1 | (1.0%) | 1 | (1.0%) | 2 | (0.7%) |

| | | | | | | |
|---|---|---|---|---|---|---|
| Other | 0 | 1 | (1.0%) | 0 | 1 | (0.3%) |

TABLE 2
Phase 2b AE Summary

| | Reproxalap 0.1% | | Reproxalap 0.25% | | Vehicle | | All Subjects | |
|---|---|---|---|---|---|---|---|---|
| | (N = 100) | | (N = 100) | | (N = 100) | | (N = 300) | |
| Number of Ocular TEAEs | 47 | | 111 | | 15 | | 173 | |
| Number of Subjects with Ocular TEAEs | 38 | (41.0%) | 93 | (93.0%) | 13 | (13.0%) | 144 | (48.0%) |
| Mild | 37 | (37.0%) | 84 | (84.0%) | 12 | (12.0%) | 133 | (44.3%) |
| Moderate | 1 | (1.0%) | 8 | (8.0%) | 1 | (1.0%) | 10 | (3.3%) |
| Severe | 0 | | 1 | (1.0%) | 0 | | 1 | (0.3%) |
| Instillation Site Pain Total | 37 | (37.0%) | 93 | (93.0%) | 2 | (2.0%) | 132 | (44.0%) |
| Prior to Day 15 | 31 | (31.0%) | 89 | (89.0%) | 1 | (1.0%) | 121 | (40.3%) |

| | | | | | | | |
|---|---|---|---|---|---|---|---|
| Day 15-Day 28 | 4 | (4.0%) | 4 | (4.0%) | 0 | 8 | (2.7%) |

| | | | | | | | |
|---|---|---|---|---|---|---|---|
| Day 29-Day 56 | 2 | (2.0%) | 0 | 1 | (1.0%) | 3 | (1.0%) |

| After Day 56 | 0 | 0 | 0 | 0 | |
|---|---|---|---|---|---|
| Number of Subjects with TEAEs | 1 | 10 | | | |
| leading to Discontinuation | | | | | |
| Prior to Day 15 | 1* | 7** | | | |
| Day 15-Day 28 | 0 | 2*** | 0 | 11 | (3.7%) |
| Day 29-Day 56 | 0 | 0 | | | |
| After Day 56 | 0 | 1 (SAE) | | | |
| Number of SAEs | 1 (peripheral | 1 (chest pain, | 0 | 2 | (0.7%) |
| | vertigo, not | not related) | | | |
| | related, stayed | | | | |
| | on study) | | | | |

*Subject discontinued at Day 12
**Subjects discontinued on the following Days: 2, 3, 5, 5, 5, 12, 14
***Subjects discontinued on the following Days: 15, 16

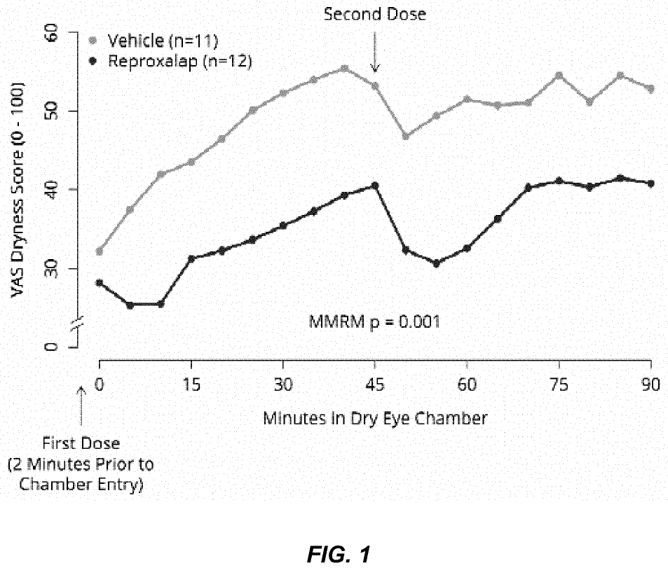

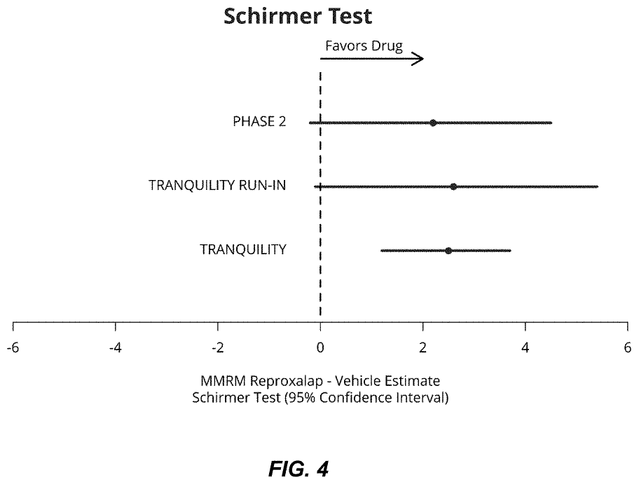

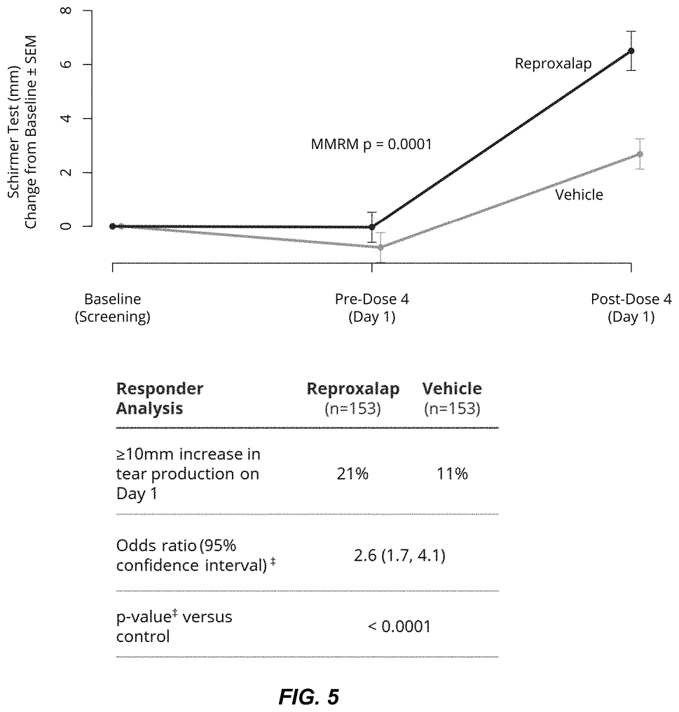

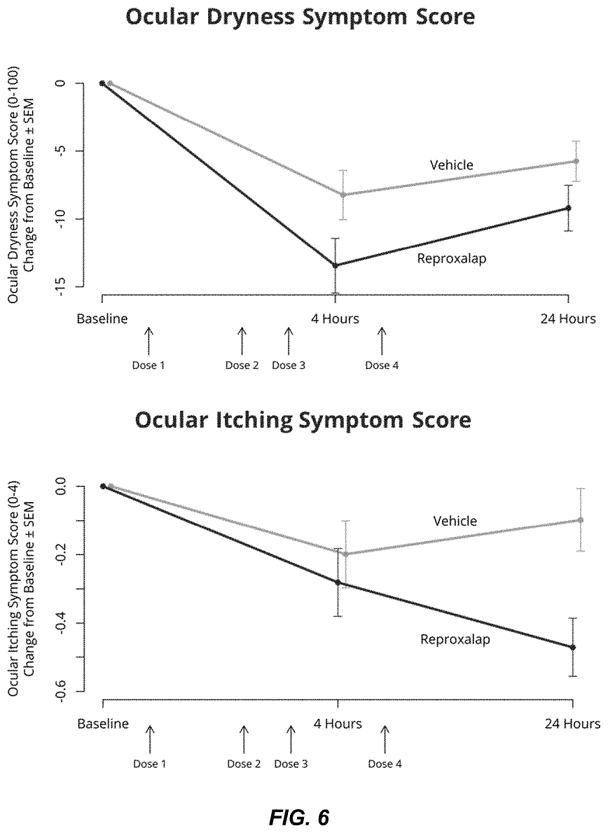

The phase 2b data are shown in FIGS. 1 through 9 and Tables 1 through 3 of International patent publication WO2020068986, incorporated herein by reference.

Key Observations From Phase 2b Clinical Trial
1. Early onset of effect from Phase 2b evidenced across multiple signs and symptoms
   Majority (>50-100%) of effect vs vehicle seen at the first study endpoint (Week 2 or 4) in 0.25% group:
      Positive early onset for 3 out of 4 symptom endpoints: ODS, OD4SQ, OSDI
         Negative for SANDE
      Positive early onset for 3 out of 4 sign endpoints: Lissamine green total score, fluorescein total score, tear osmolarity
         Negative for TFBUT® (met definition at week 4)
         Schirmer's Test only assessed at week 12
2. Dose response was demonstrated between 0.1% and 0.25% dose strengths
3. 0.1% reproxalap matched higher dose effects at later time points
   Clearest effect with signs, especially ocular staining
   Compliance poorest in 0.25% group (8% non-compliant vs 3% in the 0.1% group and 1% in the vehicle group)
4. Vehicle effect increased with study duration
   Clearest effect was observed with signs, especially ocular staining
   Normal pattern in DED with plateau around two to three months
   QID vehicle in Phase 2b was expected to have increased this effect.

### Example 2. Clinical Study to Assess the Safety and Efficacy of Reproxalap Ophthalmic Solution in a Controlled Adverse Environment (CAE®)

The objective of the study is to evaluate the efficacy of reproxalap, as assessed by tear reactive aldehyde species (RASP) levels, after single and multiple doses in subjects with dry eye disease. The efficacy of reproxalap will also be assessed by tear RASP levels, conjunctival redness, Schirmer's Test, and dry eye symptoms after dosing immediately prior to and during exposure to the Controlled Adverse Environment (CAE®) over 2 hours, in subjects with dry eye disease. CAE® chambers allow standardization of variables, such as temperature, humidity, airflow, and visual tasking (see Calonge et al., Current Eye Res., 2018, 43(4):445-450; see also Ousler, III et al., Ophthalmol Ther., 2017, 6(2): 263-276)

Dosage/Dose Regimen. A Reproxalap solution (0.25% w/v or 0.1% w/v) is administered topically to both eyes. The control is a Vehicle Control Solution. The test article is administered QID on Day 1 (Visit 2). On Day 2 (Visit 3), test article is administered once 2 or 5 minutes immediately prior to the CAE®, once 45 minutes after initiation of the CAE®, and once at CAE® exit.

Visit Schedule. Three visits are scheduled over the course of approximately 2 weeks:
Visit 1=Day −14±2, Screening
Visit 2=Day 1, Randomization/Baseline
Visit 3=Day 2 CAE® and Study Exit

Inclusion Criteria. Subjects must meet all of the following criteria for the study:
1. 18 years of age (either gender and any race);
2. Ability to provide written informed consent and sign the Health Information Portability and Accountability Act (HIPAA) form;
3. Reported history of dry eye for at least 6 months prior to Visit 1;
4. Reported history of use or desire to use eye drops for dry eye symptoms within 6 months of Visit 1;
5. Have a corneal fluorescein staining sum (sum of inferior, superior, and central) ≥4 in at least one eye on the Ora Calibra Scale;
6. Schirmer test score ≤10 mm; and
7. Response to the CAE® at Visit 1, as defined by: A ≥15-point increase in the visual analog scale eye dryness score (0-100) and a 1-point increase in ocular redness score in both eyes during at least two consecutive time points in CAE® following administration of vehicle just prior to CAE exposure and 45 minutes after CAE entry.

Exclusion Criteria. Subjects must not meet any of the following criteria:
1. Clinically significant slit lamp findings at Visit 1 that may include active blepharitis, meibomian gland dysfunction (MGD), lid margin inflammation, or active ocular allergies that require therapeutic treatment, and/or in the opinion of the investigator may interfere with study parameters;
2. Diagnosis of an ongoing ocular infection (bacterial, viral, or fungal), or active ocular inflammation at Visit 1;
3. Contact lens use within 7 days of Visit 1 or anticipate using contact lenses during the trial;
4. Eye drop use within 2 hours of Visit 1;
5. Previous laser-assisted in situ keratomileusis (LASIK) surgery within the last 12 months;
6. Cyclosporine 0.05% or 0.09% or lifitegrast 5.0% ophthalmic solution use within 90 days of Visit 1;
7. Systemic corticosteroid or other immunomodulator therapy (not including inhaled corticosteroids) within 14 days of Visit 1 or anticipate such therapy throughout the study period;
8. Planned ocular and/or lid surgeries over the study period or any ocular surgery within 6 months of Visit 1;
9. Temporary punctal plugs during the study that have not been stable within 30 days of Visit 1;
10. Use of and unwillingness to discontinue topical ophthalmic prescription (including medications for glaucoma) or over-the-counter (OTC) solutions, artificial tears, gels, or scrubs for the duration of the trial (excluding medications allowed for the conduct of the trial);
11. Corrected visual acuity greater than or equal to logarithm of the minimum angle of resolution (log MAR)+0.7 as assessed by Early Treatment of Diabetic Retinopathy Study (ETDRS) scale in both eyes at Visit 1;
12. Pregnancy, nursing, or planned pregnancy during the conduct of the trial;
13. Unwillingness to submit a urine pregnancy test at Visit 1 and Visit 4 (or early termination visit) if of childbearing potential. (Non-childbearing potential is defined as a woman who is permanently sterilized [e.g., has had a hysterectomy or tubal ligation], or is post-menopausal [without menses for 12 consecutive months]);
14. If of childbearing potential, unwillingness to use an acceptable means of birth control. (Acceptable methods of contraception include: hormonal—oral, implantable, injectable, or transdermal contraceptives; mechanical—spermicide in conjunction with a barrier such as a diaphragm or condom; intrauterine device [IUD]; or surgical sterilization of partner. For non-sexually active males or females, abstinence may be regarded as an adequate method of birth control; however, if the subject becomes sexually active during the study, he/she must agree to use adequate birth control as defined above for the remainder of the trial.);
15. Known allergy and/or sensitivity to the test article or its components;
16. A condition that the investigator feels may put the subject at significant risk, may confound the study results, or may interfere significantly with the subject's participation in the trial;
17. Current enrollment in an investigational drug or device study or have used an investigational drug or device within 30 days of Visit 1;
18. Use of reproxalap ophthalmic solution in the past year;
19. Current use of any medication known to cause ocular drying that is not used on a stable dosing regimen for at least 30 days prior to Visit 1; and
20. Inability or unwillingness to follow instructions, including participation in all study assessments and visits.

The primary endpoint is the change in levels of reactive aldehyde species (RASP) in tears. Secondary endpoints include the following:
Visual analog scale eye dryness score assessed over 90 minutes in CAE®.
Ora Calibra Ocular Discomfort Scale assessed over 90 minutes in CAE®.
Conjunctival redness assessed at over 90 minutes via digital photography in the CAE® (Key Secondary Sign Endpoint).
Schirmer's Test assessed 10 minutes after dosing.
Post-CAE® RASP change from baseline.

Safety endpoints examined are as follows:
Visual acuity
Slit-lamp evaluation
Adverse event query
Intraocular Pressure (IOP)
Dilated fundoscopy

General Statistical Methods and Types of Analyses. The primary endpoint and secondary endpoints of Schirmer's Test and post-CAE® RASP is assessed via analysis of covariance (ANCOVA), with baseline score as a covariate.

Secondary endpoints of conjunctival redness and symptoms in the CAE® is assessed via mixed effect model for repeated measures (MMRM), with baseline score as a covariate, and time and test article group as factors.

Safety endpoints will be summarized using descriptive statistics.

Initial Cohort of the trial will enroll twenty subject who meet the inclusion criteria. The Initial Cohort phase will be limited to Visit 1 (Screening), Visit 2 (Day 1), and Visit 3 (Day 2). At Day 1, evaluations include Dry Eye symptoms, Schirmer's test, and tear RASP levels. At Day 2 (Chamber test), evaluations include Dry Eye symptoms, Ocular Redness, and tear RASP levels.

In one of the CAE® studies, one dose is given 2 min prior to chamber entry. Another dose is administered 45 min after chamber entry. In some instances, subjects receive one day prior to chamber entry. Thus, some subjects receive: one dose on day prior to, one dose immediately before, and one dose 45 min after entry into a 90 min dry eye chamber with minimal humidity, high airflow, and forced visual tasking. In some instances, the subjects are given a dose at exit of the CAE®.

Initial Results. Reproxalap demonstrated rapid and broad improvements after one day (Day 1) of treatment. The evaluations of dry eye symptoms at Day 1 are given below.

TABLE

| | Change From Baseline | | |
|---|---|---|---|
| | Reproxalap | Vehicle | |
| Dry Eye Assessment (Scale) | (N = 12) | (N = 11) | P-Value |
| VAS Dryness (0-100) | −26 | +2 | 0.003 |
| OD4S: Discomfort (0-5) | −0.7 | +0.4 | 0.003 |
| OD4S: Dryness (0-5) | −1.2 | +0.1 | 0.006 |
| OD4S: Grittiness (0-5) | −1.1 | +0.1 | 0.006 |
| OD4S: Burn (0-5) | −0.1 | +0.8 | 0.07 |
| OD4S: Sting (0-5) | −0.1 | +0.4 | 0.23 |
| Ocular Discomfort Scale (0-4) | −0.7 | +0.4 | 0.07 |
| Schirmer's Test (mm)* | +3.4 | +1.3 | 0.30 |

VAS = Visual Analog Scale
QD4S = Ocular Discomfort & 4-Symptom Questionnaire
QID = Four times daily

Day 1 Schirmer's Test results is based on improvement after a single dose; all other Day 1 assessments were performed over 24 hours of QID dosing. Topical ocular reproxalap has been studied in over 1,100 patients thus far with no observed safety concerns; mild instillation site irritation is the most commonly reported adverse event in clinical trials.

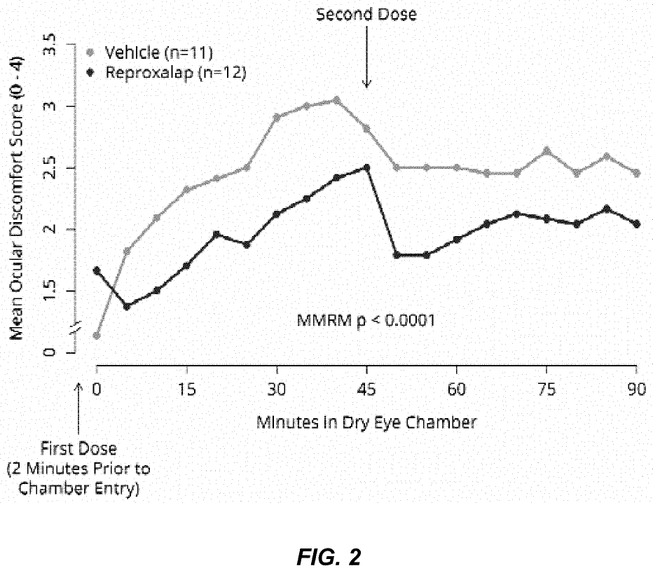

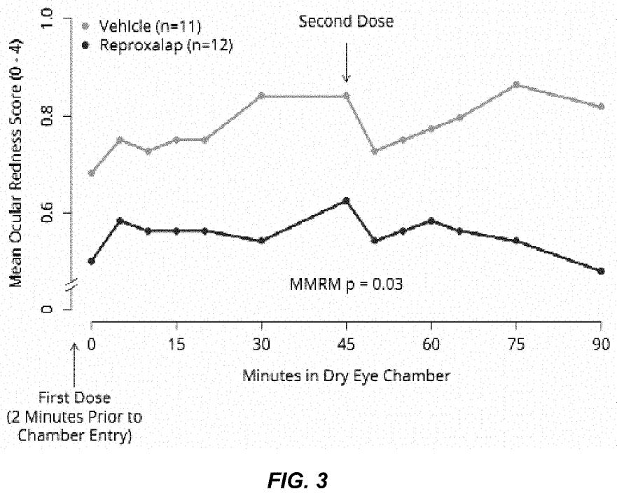

The VAS Dryness Score at Day 2 is shown in FIG. 1; the Mean Ocular Discomfort Score at Day 2 is shown in FIG. 2; and the Mean Ocular Redness Score is shown in FIG. 3. In the foregoing figures, VAS=Visual Analog Scale, and MMRM=Mixed Model Repeated Measures.

Over all time points in aggregate in the dry eye chamber studies, reproxalap was statistically superior to vehicle for the two assessed symptoms, visual analog scale (VAS) ocular dryness (p=0.001) and ocular discomfort score (p<0.0001) in the initial cohort. Reproxalap demonstrated statistically significant improvement over vehicle (p=0.03) in ocular redness, an objective sign of dry eye disease. Improvement in ocular symptoms and redness occurred within minutes following reproxalap dosing. Following acute dosing on the day prior to the dry eye chamber, Schirmer test scores were directionally in favor of reproxalap over vehicle, and reproxalap was statistically superior to vehicle in improvement in VAS dryness score (p=0.003), ocular discomfort 4-symptom questionnaire (OD4SQ) dryness score (p=0.006), OD4SQ grittiness score (p=0.006), and OD4SQ discomfort score (p=0.003).

### Example 3. Measurement of Aldehyde Biomarkers in Tear Film in Patients in Clinical Trials

The study was to assess levels of malondialdehyde (MDA) adducts in tears collected from subjects with dry eye disease enrolled in a Phase 2a, randomized, double-masked, clinical study to assess the safety, tolerability, and pharmacodynamic activity of ADX-102 ophthalmic solution in subjects with dry eye diseases, also referred to herein as dry eye syndrome (DES).

Tears were collected during Visit 1, prior to treatment, and during Visit 3, following four weeks of treatment. Treatment groups consisted of ADX-102 Ophthalmic Solution (0.5%), ADX-102 Ophthalmic Solution (0.1%), and ADX-102 Ophthalmic Lipid Solution (0.5%).

Based on results from a prior study in which normal human tear (NHT) were diluted 1:20, resulting in readings in the linear range of a standard curve, 1:20 and 1:80 dilutions in D/C subject tear samples and NHT were tested to provide reference points for MDA adduct concentrations in tears from DES and normal subjects. In the prior study, the 1:80 dilution of the high concentration spiked control sample (20,000 pmol/mL) resulted in an OD value within the standard curve (data not shown). It was anticipated that tears from DES subjects would contain similar concentrations of MDA adducts, and therefore a 1:80 dilution would result in readings in the linear range of the standard curve.

The pilot assay was conducted using 1:20 and 1:80 dilution of samples, in duplicate. Data from the pilot study showed that OD values from the D/C subject tears diluted 1:20 and 1:80 were within the linear range of the standard curve.

The pilot assay was conducted using 1:20 and 1:80 dilution of samples, in duplicate. Based on the pilot assay data, the DES subject study samples were diluted 1:60 to maximize the likelihood of the OD values falling within the linear portion of the standard curve, and thus provide the most accurate results.

Human tears were collected from subjects with DES during the Phase 2a clinical trial, according to the schedule in the table below. Tears (up to 10 μL) from both eyes of each subject were collected and pooled at Visit 1. At Visit 3, after 28 days of treatment with ADX-102 Ophthalmic Solution, human tears were collected from both eyes of each subject still enrolled. The Visit 3 tear samples from both eyes of each subject were not pooled at the time of collection, which was not in accordance with the Tear Collection Procedure Manual. Instead, right eye and left eye samples from each subject were pooled at the time of analysis for MDA adducts. Out of the 51 enrolled subjects, 12 subjects dropped out before Visit 3 tear collection and were considered D/C subjects. Tears collected at Visit 1 from D/C subjects were used in MDA adduct assay method development.

| | Order of Events |
|---|---|
| Visit 1 | Informed Consent Demographics, Medical/Medication & Ocular |
| (Day 1): | History |
| Screening and | Pregnancy Testing |
| Enrollment | Acuity Slit Lamp Exam, Tear Osmolarity |
| | Tear Collection |
| | TFBUT |
| | Fluorescein and Lissamine Staining |
| | Schirmer's Test Safety Assessments Randomization |
| | In-Office Dose & Drop Comfort Questionnaire Study Drug Dispensed |
| Visit 2 | Collection of Study Drug Medical/Medication Update: AE Query |
| (Day8 ± 1): | Visual Acuity |
| 1-Week Follow-Up | Slit Lamp Exam TFBUT, Fluorescein Staining |
| | Safety Assessments |
| | In-Office Dose & Drop Comfort Questionnaire Study Drug Dispensed |
| Visit 3 | Collection of Study Drug Medical/Medication Update: AE Query Tear |
| (Day 29 ± 2): | Symptom Questionnaires, Visual Acuity Slit Lamp Exam |
| 4-Week Follow-Up and | Tear Osmolarity |
| Study Exit | Tear Collection |
| | TFBUT, Fluorescein and Lissamine Staining, Schirmer's Test |
| | Safety Assessments, Study Exit |

Fifty-one subjects were enrolled, for a total of 17 subjects per trial arm. Subjects were randomized 1:1:1 to receive ADX-102 Ophthalmic Solution (0.10%), ADX-102 Ophthalmic Solution (0.5%), or ADX-102 Ophthalmic Lipid Solution (0.5%). A vehicle control was not included in the clinical trial.

Thirty-nine subjects completed the trial: 16 subjects in the ADX-102 Ophthalmic Solution (0.1%) group; 12 subjects in the ADX-102 Ophthalmic Solution (0.5%) group; and 11 subjects in the ADX-102 Ophthalmic Lipid Solution (0.5%) group. Twelve subjects did not complete the study (D/C subjects).

Subjects self-administered ADX-102 Ophthalmic Solution four times per day (morning, noon, afternoon, and before bed) throughout the study. Subjects did not use study drug prior to Study Visits.

MDA adduct ELISA. Normal human tears, pooled from three individuals (two males and one female), were purchased from Bioreclamation IVT (catalog number hmtears). The MDA adduct ELISA kit is commercially available and was purchased from Cell Biolabs, Inc., San Diego, Calif. (OxiSelect MDA Adduct Competitive ELISA, catalog number STA-832).

The assay is a competitive ELISA. An MDA conjugate is adsorbed onto an ELISA plate. Samples containing unknown amounts of MDA adducts or MDA-BSA standards are then added to the plate and incubated. An MDA antibody is then added to the plate, followed by an HRP-labelled secondary antibody. The plate is washed and an HRP detection agent is added. The plate is read in a microplate reader at 450 nm. The assay OD reading decreases with increasing MDA adducts in the samples, as the adsorbed MDA competes for binding to the MDA antibody with MDA adducts in the test sample.

A standard curve for the assay was generated using 0, 0.025, 0.05, 0.10, 0.20, 0.39, 0.78, 1.56, 3.13, and 6.25 μg/mL of MDA-BSA. Standard and unknown samples volumes in the assay were 50 μL each.

Pilot Assay. In addition to the standard curve, neat NHT samples and NHT samples spiked with MDA-BSA were measured. NHT spiked with 12,000 pmol/mL concentration of the internal standard, MDA-BSA, was used to determine dilutional integrity. D/C samples and NHT spiked samples were diluted 20- and 80-fold in PBS buffer containing 0.1% BSA prior to assay. Neat NHT samples were diluted 20-fold to serve as a baseline reference.

DES Study Samples. Based on the results of the pilot assay, a 1:60 dilution was determined to maximize the likelihood of the OD values falling within the linear portion of the standard curve, and thus provide the most accurate results. All tear samples from subjects (0.5% ADX-102 Ophthalmic Solution) and (0.1% ADX-102 Ophthalmic Solution) were analyzed, but results from some tear samples were excluded from the data analysis due to contamination with ocular staining dye in the Visit 3 samples. The Visit 3 tear sample from some subjects (0.5% ADX-102 Ophthalmic Solution) was excluded from the data analysis due to insufficient volume.

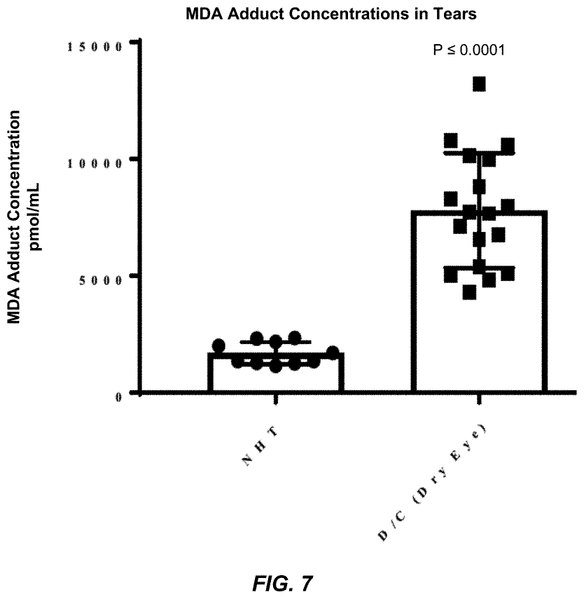

Results—Pilot assay of D/C subject and NHT. The detection range of the assay was 6 to 1500 nM, and the linear range of the assay was approximately 10 to 110 nM. Normal human tears diluted 20-fold had an average calculated MDA adduct concentration of 2,266 pmol/mL (2,266 nM). Approximately 100% of the spiked MDA-BSA adduct (12,000 pmol/mL) was recovered in NHT. Tears from D/C DES subjects had a mean MDA adduct concentration of 7,798 pmol/mL, a 3.4-fold increase relative to NHT; this difference was statistically significant (FIG. 7).

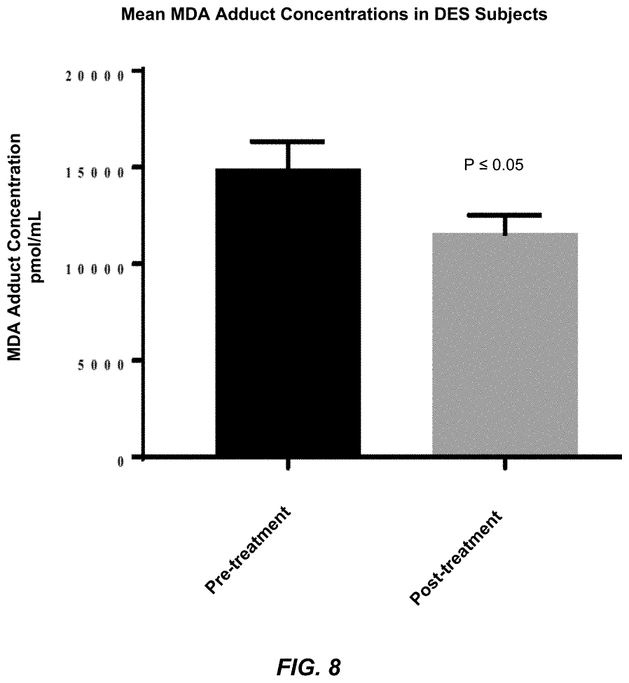

Assay of tears from subjects who completed the trial. FIG. 8 shows the calculated mean MDA adduct concentrations in tears from 37 DES subjects who completed the trial, at Visit 1 (baseline, before treatment) and Visit 3 (after 28 days of treatment with ADX-102 Ophthalmic Solution). Two subjects were excluded from this analysis because corneal fluorescein staining was conducted prior to tear collection, which interfered with the MDA adduct ELISA signal. Tears collected at Visit 1 had a mean MDA adduct concentration of 14,943 pmol/mL, which is significantly higher than the mean MDA adduct concentration of 11,566 pmol/mL in tears collected from all subjects at Visit 3.

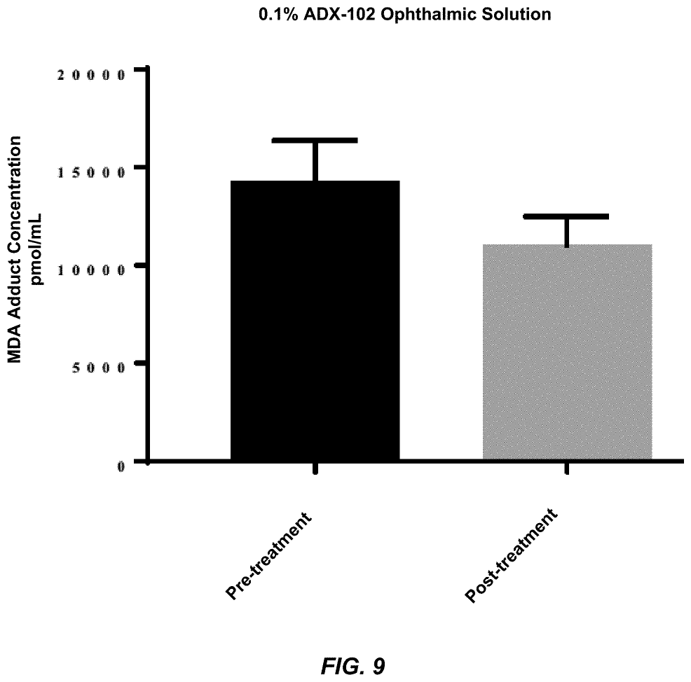

FIG. 9 shows MDA adduct concentrations in tears in subjects treated with 0.1% w/v ADX-102 Ophthalmic Solution Subjects treated with ADX-102 Ophthalmic Solution (0.1%) had a mean MDA adduct concentration of 14,287 pmol/mL at Visit 1, compared to 11,028 pmol/mL at Visit 3, which corresponds to a 23% reduction in MDA adduct levels after treatment.

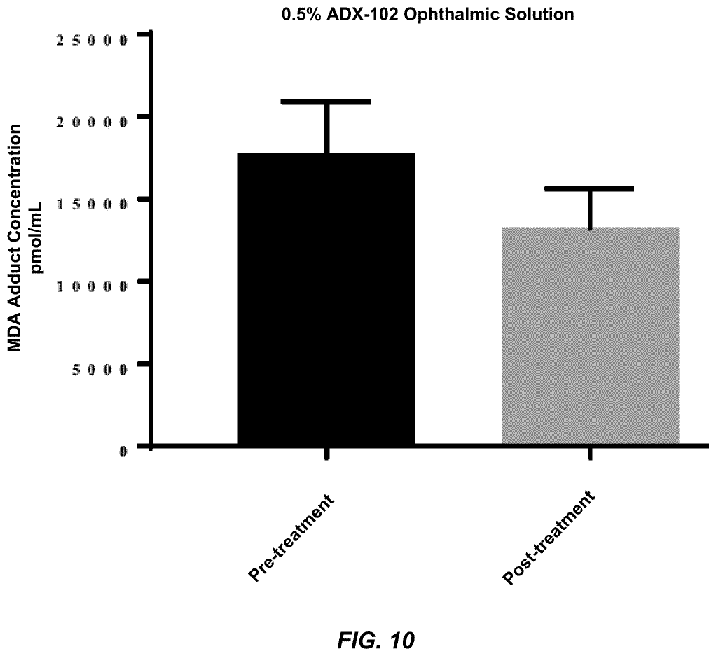

FIG. 10 shows MDA adduct concentrations in tears in subjects treated with ADX-102 Ophthalmic Solution (0.5%). Subjects treated with ADX-102 Ophthalmic Solution (0.5%) showed a 26% reduction in MDA adduct concentration at Visit 3 compared to Visit 1.

Although individually all treatment groups had lower MDA adduct concentrations on Visit 3 compared to Visit 1, the differences were not statistically significant.

Discussion MDA adducts were detected in tear samples collected from all DES subjects at Visit 1 and at Visit 3. MDA adduct concentrations were significantly lower on Visit 3 after a 4-week treatment with ADX-102 Ophthalmic Solution, relative to pre-treatment values on Visit 1, as shown in FIG. 7. Within each treatment group, MDA adduct levels in tears decreased on Visit 3 relative to Visit 1, but did not reach statistical significance. This finding may be related to the high variability in MDA adduct concentrations in DES subjects before treatment, combined with the small sample number. In addition, since the time between the last administration of study drug and collection of tears varied among subjects (estimated to be approximately eight to 12 hours), the timing of sample collection, relative to last treatment, may introduce variability into the post-treatment results at Visit 3.

The data show that MDA adduct levels are significantly higher in subjects with DES and are consistent with literature describing elevated MDA levels in tears of DES patients, compared to normal subjects. Furthermore, the data suggest that treatment with ADX-102 Ophthalmic Solution decreases MDA adducts in tears from subjects with DES.

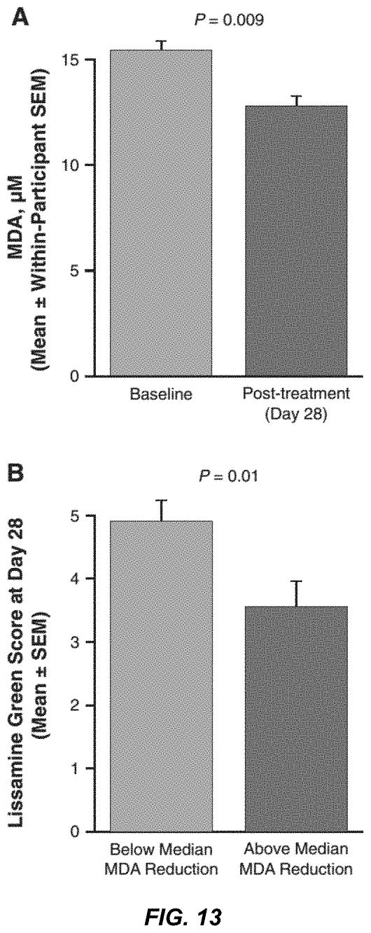

In addition, we have observed in previous Phase 2a studies that tear levels of MDA adduct were statistically lower after treatment (FIG. 13A). Participants with above-median reduction in MDA demonstrated statistically lower lissamine green staining scores than did participants with below-median reduction in MDA (FIG. 13B). Participants with above-median reduction in MDA adduct levels demonstrated statistically different tear osmolarity scores versus 0, whereas participants with below median MDA adduct reduction were not statistically different from 0. Supportive of the relationship of MDA adducts to osmolarity, reduction in osmolarity was correlated with reduction in MDA adduct levels (Pearson r=0.31, P=0.07). Results from the pooled reproxalap groups indicated that levels of MDA, a RASP previously described to be elevated in the tears of patients with DED, were statistically lower after 28 days of therapy than at baseline. Consistent with the clinical relevance of RASP as a proinflammatory mediator, reduction in MDA levels correlated with improvements in tear osmolarity and lissamine green staining. RASP are upstream pre-cytokine potentiators of the innate immune response, including activation of NF-kB, inflammasomes, and scavenger receptor A, which may broadly exacerbate anterior segment inflammatory disease. Thus, RASP inhibition could explain the multifaceted activity of reproxalap observed across several signs and symptoms of DED. To our knowledge, the MDA findings represent the first direct clinical measurement of drug mechanism of action for any DED drug.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

All publications, patents, patent applications and other documents cited in this application are hereby incorporated by reference in their entireties for all purposes to the same extent as if each individual publication, patent, patent application or other document were individually indicated to be incorporated by reference for all purposes.

We claim:

1. A method of treating dry eye disease, comprising administering to a subject in need thereof a therapeutically effective amount of an ophthalmic solution comprising reproxalap, or a pharmaceutically acceptable salt thereof, wherein the ophthalmic solution is administered topically to an eye of the subject in need thereof prior to an expected or certain exposure to one or more conditions that initiate dry eye disease or its symptoms and subsequent to exposure to conditions that initiate dry eye disease or its symptoms; wherein the method comprises an improvement in ocular dryness and redness, and/or tearing.

2. The method of claim 1, wherein the ophthalmic solution is administered four times a day (QID) in an initiation phase one day prior to the expected or certain exposure to one or more conditions that initiate dry eye disease or its symptoms followed by administration two times a day (BID) after exposure to the one or more conditions that initiate dry eye disease or its symptoms.

3. The method of claim 1, wherein the ophthalmic solution is administered four times a day (QID) in an initiation phase one day prior to the expected or certain exposure to one or more conditions that initiate dry eye disease or its symptoms followed by administration as needed (PRN) after exposure to the one or more conditions that initiate dry eye disease or its symptoms.

4. The method of claim 1, further comprising assessing effectiveness of treatment by determining one or more of ocular itchiness, burning, stinging, grittiness, cloudy vision, sensitivity to environment, and level of RASP in tears.

5. The method of claim 4, wherein assessing effectiveness of treatment comprises measuring level of RASP in tears.

6. The method of claim 5, wherein the effectiveness comprises reducing levels of RASP to levels present in normal eyes.

7. The method of claim 1, wherein the method produces an improvement in tear quantity and/or quality in the eye of the subject.

8. The method of claim 7, wherein the improvement is measured using Schirmer's Test and the Schirmer's Test score improves to at least 10 mm.

9. The method of claim 7, wherein the method produces an improvement in ocular redness.

10. The method of claim 9, wherein the method produces an improvement in ocular dryness.

11. The method of claim 10, wherein the method produces an improvement in ocular itchiness.

12. The method of claim 1, wherein the ophthalmic solution comprising reproxalap, or a pharmaceutically acceptable salt thereof, comprises reproxalap at about 0.2% w/v to about 0.3% w/v, and a pharmaceutically acceptable excipient comprising a cyclodextrin, wherein the cyclodextrin is sulfobutylether-β-cyclodextrin or hydroxypropyl-β-cyclodextrin, or a pharmaceutically acceptable salt thereof, wherein the reproxalap and cyclodextrin are present in a ratio of about 1:2, about 1:3, about 1:4, or about 1:5 on a mole:mole basis.

13. The method of claim 12, wherein reproxalap, or a pharmaceutically acceptable salt thereof, is at about 0.25% w/v.

14. The method of claim 12, wherein the cyclodextrin is sulfobutylether-β-cyclodextrin, or a pharmaceutically acceptable salt thereof.

15. The method of claim 14, wherein the reproxalap and cyclodextrin are present in a ratio of about 1:3 on a mole:mole basis.

16. The method of claim 14, wherein the reproxalap and cyclodextrin are present in a ratio of about 1:5 on a mole:mole basis.

17. The method of claim 14, wherein reproxalap, or a pharmaceutically acceptable salt thereof, is at about 0.25% w/v, and the cyclodextrin is sulfobutylether-β-cyclodextrin at about 7% w/v.

18. The method of claim 14, wherein reproxalap, or a pharmaceutically acceptable salt thereof, is at about 0.25% w/v, and the cyclodextrin is sulfobutylether-β-cyclodextrin at about 11% w/v.

19. The method of claim 14, wherein the buffering agent comprises a phosphate buffer and is at a pH of about 6.5 to about 7.5.

20. The method of claim 1, wherein the ophthalmic solution is topically administered to the eye five days or less prior to the expected or certain exposure to one or more conditions that initiate dry eye disease or its symptoms.

21. The method of claim 1, wherein the ophthalmic solution is topically administered to the eye two days or less prior to the expected or certain exposure to one or more conditions that initiate dry eye disease or its symptoms.

22. The method of claim 1, wherein the ophthalmic solution is topically administered to the eye one day or less prior to the expected or certain exposure to one or more conditions that initiate dry eye disease or its symptoms.

23. The method of claim 1, wherein the ophthalmic solution is topically administered to the eye immediately prior to the expected or certain exposure to one or more conditions that initiate dry eye disease or its symptoms.

24. The method of claim 1, wherein the ophthalmic solution is topically administered to the eye two days or less and immediately prior to the expected or certain exposure to one or more conditions that initiate dry eye disease or its symptoms.

25. The method of claim 1, wherein the ophthalmic solution is topically administered to the eye one day or less and immediately prior to the expected or certain exposure to one or more conditions that initiate dry eye disease or its symptoms.

26. The method of claim 1, wherein the ophthalmic solution is topically administered to the eye at two days prior, one day prior, and immediately prior to the expected or certain exposure to one or more conditions that initiate dry eye disease or its symptoms.

27. The method of claim 1, wherein the ophthalmic solution is administered four times or two times a day at two days prior to the expected or certain exposure to one or more conditions that initiate dry eye disease or its symptoms.

28. The method of claim 1, wherein the ophthalmic solution is administered four times or two times a day at one day prior to the expected or certain exposure to one or more conditions that initiate dry eye disease or its symptoms.

29. The method of claim 1, further comprising administering the ophthalmic solution to the eye about 45 min or 60 min after the exposure to one or more conditions that initiate dry eye disease or its symptoms, or about 45 min or 60 min after an administration immediately prior to the expected or certain exposure to one or more one or more conditions that initiate dry eye disease or its symptoms.
